(19) United States
(12) Patent Application Publication
(10) Pub. No.: US 2014/0073575 A1
(43) Pub. Date: Mar. 13, 2014

(54) COLLAGEN HYDROXYLASES

(71) Applicants: Thierry Hennet, Otelfingen (CH); Christoph Rutschmann, Zuerich (CH); Kelvin Luther, Zuerich (CH)

(72) Inventors: Thierry Hennet, Otelfingen (CH); Christoph Rutschmann, Zuerich (CH); Kelvin Luther, Zuerich (CH)

(73) Assignee: UNIVERSITAET ZUERICH PROREKTORAT MNW, Zuerich (CH)

(21) Appl. No.: 14/114,754

(22) PCT Filed: Mar. 21, 2012
(86) PCT No.: PCT/EP2012/055030

§ 371 Date: Oct. 30, 2013

(30) Foreign Application Priority Data
Apr. 15, 2011 (EP) 11162760.0

(51) Int. Cl. C07K 14/78; C12N 9/02

(52) U.S. Cl. CPC C07K 14/78; C12N 9/0071
USPC 514/17.2; 435/68.1; 536/23.2; 435/320.1; 435/252.33; 435/69.1; 514/44 R; 435/408; 435/254.2; 435/252.3; 435/254.11; 435/419; 435/325; 435/348

(57) ABSTRACT

Prolyl and lysyl hydroxylases isolated from Mimivirus are described. These are able to hydroxylate collagen. Isolated nucleic acids coding for the mentioned hydroxylases are incorporated into suitable vectors and used to express these hydroxylases in host cells, e.g. E. coli. Furthermore a method of manufacturing hydroxylated collagen in a host cell is described. The hydroxylases and the recombinantly expressed hydroxylated collagen are useful in clinical settings and biotechnology applications.

### FIELD OF THE INVENTION

The invention relates to isolated prolyl and lysyl hydroxylases from Mimivirus able to hydroxylate collagen, to isolated nucleic acids coding for the mentioned hydroxylases, and to a method of manufacturing these hydroxylases and hydroxylated collagen in a host cell.

### BACKGROUND OF THE INVENTION

Collagens are the most abundant proteins in animals. In mammals up to 29 types of collagens have been identified. Collagens act not only as scaffold for tissues but also as regulators of many biological processes including cell attachment, proliferation and differentiation (Myllyharju, J. and Kivirikko, K. I. (2004) Trends Genet. 20, 33-43; Shoulders, M. D. and Raines, R. T. (2009) Annu Rev Biochem, 929-58). Structurally, collagens are subdivided into fibrillar collagens, such as types-I, II, Ill, and non-fibrillar collagens, such as types-IV, VI, XII. Collagens are mainly linear proteins characterized by domains composed mainly of repeats of the triplet Gly-X-Y, where proline and lysine are often found at positions X and Y. After synthesis in the endoplasmic reticulum, three procollagen subunits associate to build a right-handed triple helix. However, before formation of the triple helix structure, the nascent procollagen polypeptides undergo several post-translational modifications. These modifications involve the hydroxylation of selected proline and lysine residues. Some hydroxylysine (HyK) residues are further modified by the addition of carbohydrates, thus forming the collagen specific disaccharide Glc(α1-2)Gal(β1-O)HyK. The extent of HyK glycosylation varies with the types of collagen and their tissue distribution.

The formation of 4-hydroxyproline (HyP) is essential for ensuring the thermal stability of the collagen triple helix (Myllyharju, J. and Kivirikko, K. I. (2004) Trends Genet. 20, 33-43; Shoulders, M. D. and Raines, R. T. (2009) Annu Rev Biochem, 929-58). In vertebrates, prolyl 4-hydroxylase (P4H) is a tetrameric enzyme comprising two alpha and two beta units. In lower organisms and in plants, several monomeric P4H enzymes have been described, which are likely involved in the hydroxylation of proteins different from collagens. The hydroxylation of selected lysine residues is mediated by three lysyl hydroxylase enzymes in vertebrates. These monomeric enzymes differ in their substrate preferences, which result in different lysyl hydroxylation patterns in various types of collagen. Lysyl hydroxylation is important for the cross-linking of collagen fibrils in addition to serving as a substrate for glycosylation reactions.

The importance of collagen post-translational modifications is reflected in the diseases associated with defective collagen modifications. Mutations in the lysyl hydroxylase PLOD1, PLOD2 and PLOD3 genes leads to Ehlers-Danlos type-VI, Bruck syndrome and to a form of skeletal dysplasia, respectively. While the role of proline and lysine hydroxylation in ensuring collagen stability and fibril formation is well established, the functional relevance of collagen glycosylation is presently unclear. In fact, the genes encoding the collagen galactosyltransferase enzymes were only identified recently (Schegg, B., Hülsmeier, A. J., Rutschmann, C., Maag, C., and Hennet, T. (2009) Mol Cell Biol 29, 943-952). As deduced from whole genome RNA interference studies in Caenorhabditis elegans, it appears that loss of collagen galactosyltransferase is associated with severe phenotypes like slow growth, abnormal locomotion and sterility. Interestingly, glycosylated HyK also occurs in the collagen domains of non-fibrillar proteins such as the hormone adiponectin, the mannose-binding lectin and the acetylcholine esterase complex. Since the collagen domain of these proteins is involved in protein folding and oligomerization, it is likely that the glycan chains are involved in this process, too.

The formation of the triple helix begins at the C-terminus right after completion of translation. The C-terminal propeptides are cross-linked by disulfide bridges, which bring the Gly-X-Y repeat regions together, thereby creating a nucleation point for the spontaneous formation of the triple helix structure. This process requires HyP but proceeds without the involvement of chaperones. After formation of the triple helix, collagen transits through the secretory pathway. The N- and C-terminal propeptides, which are still flanking the Gly-X-Y repeat region, are important for maintaining the solubility of collagen inside the cell. Once in the extracellular space, the propeptides are cleaved by specific collagen propeptidases and lysyl oxidases initiate the formation of covalent cross-links, which stabilize collagen fibrils.

Because of the essential role of prolyl and lysyl hydroxylation, the production of recombinant collagen requires the use of expression systems that include hydroxylase activities. Such activities are only found in animal cells, thereby precluding the expression of collagen in bacterial and yeast expression systems without specific modifications. However, large-scale production of recombinant collagens is hampered in animal cells by the poor yields achieved and high costs of maintaining cells in culture. The yeast Pichia pastoris has been engineered to express the human P4H enzyme (Pakkanen, 0., Pirskanen, A., and Myllyharju, J. (2006) J Biotechnol 123, 248-256), which enables an adequate level of prolyl hydroxylation and thus ensures the formation of triple helix collagen. However, efficient lysyl hydroxylation has not been achieved in Pichia to date. The human P4H has also been expressed as active protein in the periplasm of bacteria but the ability of the enzyme to hydroxylate collagen in the bacterial expression system has not been demonstrated. So far, the co-expression of collagens and hydroxylase enzymes in bacteria has failed to yield hydroxylated recombinant collagen.

### SUMMARY OF THE INVENTION

The invention relates to isolated prolyl and lysyl hydroxylase from Mimivirus able to hydroxylate collagen.

In particular, the invention relates to an iso-
lated protein comprising the sequence
(SEQ ID NO: 1)
MVLSKSCVSHFRNVGSLNSRDVNLKDDFSYANIDDPYNKPFVLNNLINPT
KCQEIMQFANGKLFDSQVLSGTDKNIRNSQQMWISKNNPMVKPIFENICR
QFNVPFDNAEDLQVVRYLPNQYYNEHHDSCCDSSKQCSEFIERGGQRILT
VLIYLNNEFSDGHTYFPNLNQKFKPKTGDALVFYPLANNSNKCHPYSLHA
GMPVTSGEKWIANLWFRERKFS;
an isolated protein comprising the sequence
(SEQ ID NO: 2)
MKTVTIITIIVVIIVVILIIMVLSKSCVSHFRNVGSLNSRDVNLKDDFSY
ANIDDPYNKPFVLNNLINPTKCQEIMQFANGKLFDSQVLSGTDKNIRNSQ
QMWISKNNPMVKPIFENICRQFNVPFDNAEDLQVVRYLPNQYYNEHHDSC
CDSSKQCSEFIERGGQRILTVLIYLNNEFSDGHTYFPNLNQKFKPKTGDA
LVFYPLANNSNKCHPYSLHAGMPVTSGEKWIANLWFRERKFS;
an isolated protein comprising the sequence
(SEQ ID NO: 3)
MISRTYVINLARRPDKKDRILAEFLKLKEKGVELNCVIFEAVDGNNPEHL
SRFNFKIPNWTDLNSGKPMTNGEVGCALSHWSVWKDVVDCVENGTLDKDC
RILVLEDDVVFLDNFMERYQTYTSEITYNCDLLYLHRKPLNPYTETKIST
HIVKPNKSYWACAYVITYQCAKKFMNANYLENLIPSDEFIPIMHGCNVYG
FEKLFSNCEKIDCYAVQPSLVKLTSNAFNDSETFHSGSYVPSNKFNFDTD
KQFRIVYIGPTKGNSFHRFTEYCKLYLLPYKVIDEKETNDFVSLRSELQS
LSEQDLNTTLMLVVSVNHNDFCNTIPCAPTNEFIDKYKQLTTDTNSIVSA
VQNGTNKTMFIGWANKISEFINHYHQKLTESNAETDINLANLLLISSISS
DFNCVVEDVEGNLFQLINEESDIVFSTTTSRVNNKLGKTPSVLYANSDSS
VIVLNKVENYTGYGWNEYYGYHVYPVKFDVLPKIYLSIRIVKNANVTKIA
ETLDYPKELITVSISRSEHDSFYQADIQKFLLSGADYYFYISGDCIITRP
TILKELLELNKDFVGPLMRKGTESWTNYWGDIDPSNGYYKRSFDYFDIIG
RDRVGCWNVPYLASVYLIKKSVIEQVPNLFTENSHMWNGSNIDMRLCHNL
RKNNVFMYLSNLRPYGHIDDSINLEVLSGVPTEVTLYDLPTRKEEWEKKY
LHPEFLSHLQNFKDFDYTEICNDVYSFPLFTPAFCKEVIEVMDKANLWSK
GGDSYFDPRIGGVESYPTQDTQLYEVGLDKQWHYVVFNYVAPFVRHLYNN
YKTKDINLAFVVKYDMERQSELAPHHDSSTYTLNIALNEYGKEYTAGGCE
FIRHKFIWQGQKVGYATIHAGKLLAYHRALPITSGKRYILVSFVN;

and variants of such proteins comprising variants of SEQ ID NO:1, SEQ ID NO:2 or SEQ ID NO:3 in which one, two, three, four, or five amino acids are exchanged by other naturally occurring amino acids.

More particularly, the invention relates to an isolated protein comprising the sequence of SEQ ID NO:1, of SEQ ID NO:2, or of SEQ ID NO:3. Most preferred is an isolated protein of the sequence SEQ ID NO:1, SEQ ID NO:2 or SEQ ID NO:3, preferably SEQ ID NO:1 or SEQ ID NO:3.

In another aspect, the invention relates to an isolated nucleic acid coding for the mentioned proteins, e.g. for a preferred protein.

In particular the invention relates to an isolated
DNA comprising a DNA of the sequence
(SEQ ID NO: 4)
ATGGTATTGTCAAAATCTTGTGTGTCCCATTTTAGAAATGTCGGTAGTTT
GAATTCACGGGATGTGAATTTAAAAGATGATTTTTCGTACGCCAATATTG
TGATCCTTATAATAAACCATTTGTTTTGAATAATTTGATTAATCCGACCA
AATGTCAAGAAATTATGCAATTTGCCAATGGAAAACTTTTTGATTCACAA
GTTTTAAGTGGTACTGATAAAAATATTCGCAATAGTCAACAAATGTGGAT
ATCTAAAAATAATCCTATGGTCAAACCTATTTTTGAAAATATATGTAGAC
AATTTAATGTTCCATTTGATAATGCTGAAGATTTGCAAGTTGTTCGTTAT
TTACCTAATCAATATTATAATGAACACCATGATTCATGCTGTGATAGTAG
TAAACAATGTAGTGAATTTATTGAAAGAGGTGGTCAAAGAATTCTAACCG
TACTAATTTATCTCAATAATGAATTTTCTGATGGTCATACTTATTTCCCT
AATTTGAATCAAAAATTCAAACCAAAAACAGGAGATGCACTAGTATTTTA
CCCTTTAGCTAATAATAGTAATAAATGTCATCCATATTCTTTACATGCTG
GAATGCCTGTTACTAGTGGAGAAAAATGGATTGCCAATTTATGGTTTAGA
GAACGAAAATTTTCCTAA;
(SEQ ID NO: 5)
ATGAAAACTGTGACTATCATTACAATAATTGTTGTAATTATTGTTGTTAT
TTTGATTATAATGGTATTGTCAAAATCTTGTGTGTCCCATTTTAGAAATG
TCGGTAGTTTGAATTCACGGGATGTGAATTTAAAAGATGATTTTTCGTAC
GCCAATATTGATGATCCTTATAATAAACCATTTGTTTTGAATAATTTGAT
TAATCCGACCAAATGTCAAGAAATTATGCAATTTGCCAATGGAAAACTTT
TTGATTCACAAGTTTTAAGTGGTACTGATAAAAATATTCGCAATAGTCAA
CAAATGTGGATATCTAAAAATAATCCTATGGTCAAACCTATTTTTGAAAA
TATATGTAGACAATTTAATGTTCCATTTGATAATGCTGAAGATTTGCAAG
TTGTTCGTTATTTACCTAATCAATATTATAATGAACACCATGATTCATGC
TGTGATAGTAGTAAACAATGTAGTGAATTTATTGAAAGAGGTGGTCAAAG
AATTCTAACCGTACTAATTTATCTCAATAATGAATTTTCTGATGGTCATA
CTTATTTCCCTAATTTGAATCAAAAATTCAAACCAAAAACAGGAGATGCA
CTAGTATTTTACCCTTTAGCTAATAATAGTAATAAATGTCATCCATATTC
TTTACATGCTGGAATGCCTGTTACTAGTGGAGAAAAATGGATTGCCAATT
TATGGTTTAGAGAACGAAAATTTTCCTAA;
(SEQ ID NO: 6)
ATGATTAGTAGAACTTATGTAATTAATCTTGCTAGACGACCTGATAAGAA
AGATCGTATTCTTGCGGAATTCCTCAAACTCAAAGAAAAAGGTGTTGAGC
TTAATTGTGTAATTTTTGAAGCTGTTGATGGAAATAATCCGGAACATTTA
TCGAGATTTAATTTCAAGATTCCTAATTGGACTGACTTGAATTCAGGTAA
GCCAATGACTAATGGAGAAGTTGGTTGTGCATTGAGTCATTGGTCTGTGT
GGAAAGATGTTGTGGATTGTGTAGAAAATGGTACTCTAGATAAAGATTGT
CGCATTCTTGTATTGGAAGATGATGTTGTTTTTCTTGATAATTTTATGGA
ACGATATCAAACTTATACTTCTGAAATTACTTACAATTGTGATCTACTCT
ACCTGCATAGAAAACCTTTGAATCCCTATACTGAAACAAAAATCTCTACT
CATATTGTCAAACCAAATAAAAGTTACTGGGCTTGCGCATATGTCATTAC
TTATCAATGTGCCAAAAAATTCATGAATGCTAATTATTTAGAAAACCTAA
TTCCGAGTGATGAATTTATTCCGATTATGCATGGGTGTAATGTCTATGGT
TTTGAAAAATTATTTTCCAATTGTGAAAAAATAGATTGTTACGCAGTTCA
ACCTAGTCTCGTAAAATTAACATCTAATGCTTTTAATGATAGCGAAACAT
TTCATTCGGGTTCTTATGTACCAAGTAATAAATTTAATTTTGATACTGAT
AAACAGTTTAGAATTGTATATATTGGACCCACTAAAGGTAATTCATTCCA
TAGATTTACTGAATATTGTAAACTTTATTTATTACCTTATAAAGTGATCG
ATGAAAAAGAAACCAATGATTTTGTTTCACTTAGATCAGAACTTCAATCT
CTTAGTGAACAAGATCTCAATACCACACTCATGTTGGTTGTTTCAGTAAA
TCACAATGATTTTTGTAATACTATTCCATGTGCACCAACCAATGAATTCA
TTGACAAGTATAAACAATTAACAACTGATACTAATTCTATTGTTAGTGCT
GTTCAAAATGGAACTAATAAGACTATGTTCATCGGTTGGGCCAATAAAAT
TAGTGAATTTATTAATCATTATCATCAAAAACTTACTGAATCTAATGCCG
AAACAGATATTAATCTAGCTAATTTATTACTTATAAGTTCTATTTCATCC
GATTTTAATTGTGTTGTAGAAGATGTTGAAGGTAATTTGTTCCAATTAAT
TAACGAAGAATCAGATATTGTATTTAGTACAACAACTTCCAGAGTCAACA
ACAAATTAGGTAAAACACCAAGCGTTTTGTATGCCAATTCTGATTCTTCT
GTGATTGTACTTAATAAAGTAGAAAATTATACAGGTTATGGTTGGAATGA
ATATTATGGTTATCATGTTTATCCAGTTAAATTTGATGTTCTTCCAAAAA
TCTATCTTTCAATTCGCATTGTAAAGAATGCAAATGTTACTAAAATTGCT
GAAACTCTTGACTATCCAAAAGAATTAATCACTGTTTCGATCAGTCGATC
AGAACATGATAGTTTTTATCAAGCTGATATTCAGAAATTCTTATTGAGTG
GTGCTGATTATTATTTTTACATTTCAGGAGATTGTATCATTACTCGACCA
ACTATTCTAAAAGAACTTCTGGAACTCAATAAAGATTTTGTAGGTCCTCT
CATGCGTAAGGGTACTGAATCATGGACTAACTATTGGGGTGATATCGATC
CTTCTAATGGTTATTACAAAAGATCATTTGATTATTTTGATATTATTGGT
AGAGATAGAGTTGGTTGTTGGAATGTACCATATCTGGCAAGCGTCTATTT
AATTAAAAAATCTGTCATTGAACAAGTTCCAAATTTGTTTACTGAAAATA
GTCACATGTGGAATGGTAGTAATATTGATATGAGATTATGTCACAATCTT
CGTAAAAATAATGTATTCATGTATTTGAGTAATCTCCGTCCTTATGGACA
CATTGATGATTCTATTAACCTGGAAGTTCTTTCTGGTGTTCCTACCGAAG
TTACTCTTTATGATCTTCCAACGCGAAAAGAAGAATGGGAGAAAAAGTAT
CTTCATCCCGAATTTTTGAGTCATTTACAAAATTTTAAAGATTTTGATTA
TACTGAAATTTGTAACGATGTTTATAGTTTCCCACTTTTTACACCTGCTT
TCTGTAAAGAGGTTATTGAAGTTATGGATAAAGCCAATTTGTGGTCTAAA
GGTGGTGATTCTTATTTTGATCCAAGAATTGGTGGTGTTGAATCTTATCC
TACTCAAGATACTCAACTGTATGAGGTAGGATTAGATAAACAATGGCATT
ATGTCGTTTTCAATTATGTTGCACCATTTGTACGTCATTTATACAATAAT
TATAAAACCAAAGATATTAATTTAGCTTTTGTTGTTAAATATGATATGGA
AAGACAATCTGAATTGGCTCCTCATCATGATTCTTCCACATATACTTTAA
ATATTGCACTTAATGAATACGGTAAAGAATATACGGCCGGAGGTTGCGAG
TTCATTCGTCATAAATTTATCTGGCAAGGACAAAAAGTTGGTTACGCTAC
AATTCACGCTGGAAAACTATTGGCATATCATCGAGCTCTTCCAATTACTT
CCGGTAAAAGATATATTTTAGTGTCTTTTGTTAATTAA;

and variants of such DNA comprising variants of SEQ ID NO:4, SEQ ID NO:5 or SEQ ID NO:6 in which one or more, in particular between one and ten nucleotides, are replaced by other nucleotides in a triplet codon coding for the same amino acid as the original triplet codon, and/or one, two, three, four, or five, triplet codons are replaced by triplet codons coding for a different amino acid.

In another aspect the invention relates to a vector comprising a DNA as defined above, in particular a bicistronic vector comprising a DNA of the sequence SEQ ID NO:4 and of the sequence SEQ ID NO:6, and variants thereof as defined above.

In yet another aspect the invention relates to a host cell comprising a vector of the invention, and to methods of expressing a hydroxylase comprising culturing a host cell comprising a vector of the invention. In a particular embodiment, the invention relates to a host cell expressing collagen or proteins containing collagen domains, a prolyl hydroxylase of the invention, and a lysyl hydroxylase of the invention, preferably collagen, a protein comprising the sequence SEQ ID NO:1 and a protein comprising the sequence SEQ ID NO:3.

The invention further relates to the hydroxylases as defined herein for use in the hydroxylation of collagen, in particular for in situ hydroxylation of collagen in a host cell, such as a bacterial host cell, e.g. E. coli. Likewise the invention relates to the mentioned proteins and preferred proteins for use in the glycosylation of collagen, in particular for in situ glycosylation of collagen in a host cell, and to other uses, such as specific proline and lysine hydroxylation in other proteins, and in a gene therapy setting.

In a further aspect the invention relates to the manufacture of collagen and proteins comprising collagen domains in a host cell, for example in a bacterial host cell such as E. coli, comprising culturing a host cell comprising DNA coding for collagen or for a protein with a collagen domain and for a hydroxylase of the invention, to such collagens produced and to their use in various medical applications.

### BRIEF DESCRIPTION OF THE FIGURES

FIG. 1. Expression and purification of prolyl hydroxylase L593_short (L593_S) and L593_long (L593_L) in E. coli. The short and long forms of the prolyl hydroxylase L593 gene were expressed as cytoplasmic His-tagged proteins in BL21(DE3) E. coli. After lysing the cells, the recombinant L593 proteins were purified by affinity chromatography on Ni-Sepharose columns. The Coomassie stained polyacrylamide gels show various fractions collected during the purification process. CL: cell lysate, W1-W3: fractions collected during the washing of the columns with 40 mM imidazole, EL1-EL2: eluate collected after application of 200 mM imidazole, EL3: eluate collected after application of 1 M imidazole. The positions of the L593_S and L593_L proteins are marked with arrows (28 and 30 kDa).

FIG. 2. Prolyl hydroxylase activity assay. Prolyl hydroxylase activity was assayed by measuring [¹⁴C]-CO₂ production after reaction of the donor substrate [¹⁴C]-2-oxoglutarate with the acceptor peptide substrate (GPP)₇ (SEQ ID NO:7) in the presence of crude and purified recombinant L593_short (L593_S) and L593_long (L593_L) proteins. Reactions were incubated at 37° C. for 45 min. The prolyl hydroxylase activity was highest when using the purified L593_S protein. Data show the average and standard deviation of three experiments. CL: cell lysate, EL: eluate of affinity purified L593 protein, M: mock, A: activity [pmol/min/mg protein].

FIG. 3. Expression and purification of lysyl hydroxylase L230 in E. coli. The lysyl hydroxylase L230 gene was expressed as cytoplasmic His-tagged protein in BL21(DE3) E. coli. Cells were lysed and the recombinant L230 protein was purified by Ni-Sepharose chromatography. The Coomassie stained polyacrylamide gels show various fractions collected during the purification process. FT: flow-through after incubation with Ni-Sepharose, W1: MCAC10 wash containing 1 M NaCl, W2: MCAC10 wash, W3-W7: fractions from a 10 mM to 100 mM imidazole wash gradient averaging 19 mM in W3, 37 mM in W4, 55 mM in W5, 73 mM in W6, and 91 mM in W7, EL1: protein eluate from the beginning of a 100 mM to 500 mM imidazole elution gradient, EL2: major elution peak from a 100 mM to 500 mM imidazole gradient. An arrow marks the position of the L230 protein (106 kDa).

FIG. 4. Lysyl hydroxylase activity assay. Lysyl hydroxylase activity was determined using purified recombinant L230 protein produced in E. coli and the following acceptor peptide substrates. GDK: (GDK)₄ (SEQ ID NO:8), GIK: (GIK)₄ (SEQ ID NO:9), MimiA: GIMGYKGEKGEI (SEQ ID NO:10), MimiB: GDKGDVGDKGDV (SEQ ID NO:11), MimiC: GDIGSKGETGNK (SEQ ID NO:12), MimiD: GTKGETGLKGII (SEQ ID NO:13), M: mock, P: product [pmol/min/mg]. Reactions were incubated at 37° C. for 60 min. Data show the average and standard error of six experiments.

FIG. 5. Co-expression of human collagen III and Mimivirus prolyl hydroxylase L593_short in E. coli. The top panel shows a Coomassie stained polyacrylamide gel loaded with fractions collected during the affinity purification of recombinant human collagen III and recombinant Mimivirus prolyl hydroxylase L593_short from the lysate of E. coli. FT: flowthrough fraction after applying the cell lysate on the Ni-sepharose column, W1-W3: fractions collected during the washing of the columns with 40 mM imidazole, EL1-EL2: eluate collected after application of 200 mM imidazole, EL3-EL6: eluate collected after application of 1 M imidazole. The positions of the human collagen III and Mimivirus L593_short proteins are marked with arrows (>100 kDa and 28 kDa). The bottom panel shows a Western blot of some of the collected fractions shown at the top panel. An anti-His₆ monoclonal mouse antibody and a HRP-labeled goat anti-mouse IgG antibody were used as primary and secondary antibody, respectively. The positions of the human collagen III (>100 kDa) and Mimivirus L593_short (28 kDa) proteins are marked with arrows.

FIG. 6. Co-expression of human collagen III and Mimivirus lysyl hydroxylase in E. coli. The top panel shows a Ponceau Red stained PVDF membrane after transfer from a PAGE of protein fractions collected during the purification of recombinant human collagen III and recombinant Mimivirus lysyl hydroxylase L230 co-expressed in E. coli. W1: wash fraction after application of 40 mM imidazole, EL1-EL3: eluate collected after application of 200 mM imidazole, EL4-EL6: eluate collected after application of 1 M imidazole, C (Control): purified lysyl hydroxylase L230. The positions of the human collagen III (>100 kDa) and Mimivirus L230 (106 kDa) proteins are marked with arrows. The bottom panel shows a Western blot of the same PVDF membrane after reaction with anti-His₆ monoclonal antibody. The positions of the human collagen III (>100 kDa) and Mimivirus L230 (106 kDa) proteins are marked with arrows.

FIG. 7. Amino acid analysis of human collagen III co-expressed with Mimivirus prolyl hydroxylase in E. coli. Recombinant human collagen III expressed in E. coli alone or together with Mimivirus prolyl hydroxylase L593_short was affinity purified and hydrolysed to amino acids. After labeling with the fluorochrome FMOC, the amino acid composition was determined by reverse-phase HPLC. The top panel shows the retention times of amino acid standards (AA). Individual amino acids are labeled using the single letter code, whereas hydroxyproline is labeled as HyP and hydroxylysine as HyK. The middle panel shows the amino acid composition of human recombinant collagen III (C-III) co-expressed with Mimivirus prolyl hydroxylase L593_short. The peak corresponding to HyP is marked by an arrow. The bottom panel shows human recombinant collagen III (C-III) expressed alone in E. coli. No HyP can be detected.

FIG. 8. Localization of hydroxyproline residues on recombinant human collagen III co-expressed with prolyl hydroxylase L593_short in E. coli. Recombinant human collagen III was purified by affinity chromatography on Ni-Sepharose and digested with trypsin. The resulting peptides were analyzed by mass-spectrometry. The present spectrum shows a peptide containing hydroxyproline (marked as HyP). I: intensity.

FIG. 9. Amino acid analysis of human collagen III co-expressed with Mimivirus lysyl hydroxylase. Recombinant human collagen III expressed in E. coli alone or together with Mimivirus lysyl hydroxylase L230 was affinity purified and hydrolyzed to amino acids. The amino acid composition was determined by HPLC as outlined under FIG. 7.

A) The top two panels show a portion of the standard amino acid profile focusing around hydroxylysine (HyK). The third panel shows amino acids isolated from human collagen Ill co-expressed with L230 lysyl hydroxylase.
B) The top panel shows a portion of the standard amino acid profile and the bottom panel the amino acid composition of human collagen Ill expressed alone, where HyK is not detectable.

FIG. 10. Expression and purification of prolyl and lysyl hydroxylase on same expression vector co-expressed with human collagen Ill. The left panel shows a Coomassie stained gel of co-expressed collagen Ill with L230 and L593_short in E. coli, purified with Ni-Sepharose, FT: Lysate Flowthrough, W1-WX: Wash with 40 mM Imidazole, EL1-EL3: Elution with 200 mM Imidazole, EL4: Elution 1 M Imidazole, collagen Ill marked with arrow. L230 marked with arrow (106 kDa), L593_short marked with arrow (28 kDa). The right panel shows a Western blot of collected fractions WX and EL1. An anti-His₆ monoclonal mouse antibody and a HRP-labeled goat anti-mouse IgG antibody were used as primary and secondary antibody, respectively. The positions of the human collagen Ill (>110 kDa), Mimivirus L230 (106 kDa) and Mimivirus L593_short (28 kDa) proteins are marked with arrows.

FIG. 11. Amino acid analysis of human collagen III co-expressed with Mimivirus L593 prolyl hydroxylase and L230 lysyl hydroxylase. Recombinant human collagen III expressed in E. coli was affinity purified and hydrolyzed to amino acids. The amino acid composition was determined by HPLC as outlined under FIG. 7. The top two panels show the HPLC profile of a standard amino acid mixture with the amino acid peaks marked with the single letter code. Hydroxyproline (HyP) and hydroxylysine (HyK) are marked with arrows. The middle panel shows the amino acid composition of human collagen Ill co-expressed with L593 and L230. The HyP and HyK peaks are marked with arrows. The bottom panel shows the amino acid composition of human collagen Ill expressed alone in E. coli where the hydroxylated amino acids HyP and HyK are not detected.

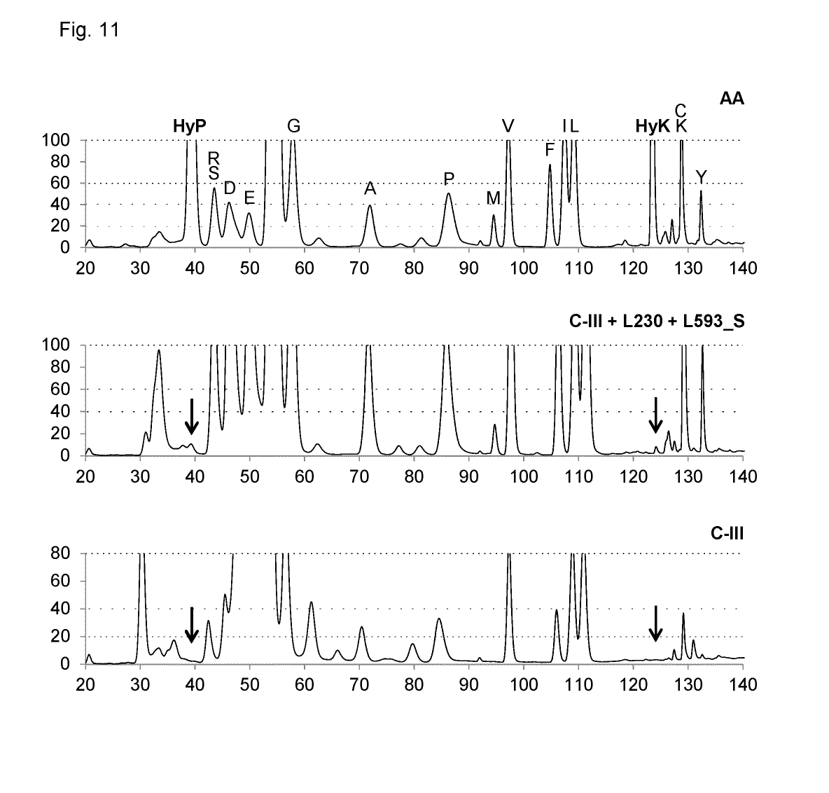

### DETAILED DESCRIPTION OF THE INVENTION

The invention relates to isolated prolyl and lysyl hydroxylase from Mimivirus able to hydroxylate collagen. Mimivirus (Acanthamoeba polyphaga mimivirus, APMV) is a giant virus, which expresses seven collagen genes and at least two collagen hydroxylases.

In particular, the invention relates to an isolated protein comprising the sequence SEQ ID NO:1; an isolated protein comprising the sequence SEQ ID NO:2; an isolated protein comprising the sequence SEQ ID NO:3; and variants of such proteins comprising variants of SEQ ID NO:1, SEQ ID NO:2 or SEQ ID NO:3 in which one, two, three, four, or five amino acids are exchanged by other naturally occurring amino acids. In particular, the invention relates to the mentioned proteins for use as a hydroxylase, such as a prolyl hydroxylase and a lysyl hydroxylase, and to a hydroxylase comprising SEQ ID NO:1, SEQ ID NO:2 or SEQ ID NO:3, or variants thereof as defined. In particular, the invention relates to a prolyl hydroxylase comprising SEQ ID NO:1 or SEQ ID NO:2 or variants thereof as defined, and to a lysyl hydroxylase comprising SEQ ID NO:3 or variants thereof as defined.

Since the sequence of SEQ ID NO:2, designated L593_long, comprises the sequence of SEQ ID NO:1 (L593_short) and in fact extends it at the N terminal by a further 20 amino acids, a protein comprising SEQ ID NO:2 likewise represents a protein comprising SEQ ID NO:1. L593_long and L593_short represent prolyl hydroxylases, as will be demonstrated further below.

The sequence of SEQ ID NO:3 is designated L230 and represents a lysyl hydroxylase, as will be demonstrated further below.

The invention relates also to variants of the mentioned sequences. Although it is well known that the substitution of one amino acid by another amino acid within the sequence of a protein may have substantial influence on the secondary, tertiary and quaternary structure of the protein, and hence on the biological function of that protein, there are substitutions which are known to have minimal influence on the biological properties, such as replacement of a non-polar amino acid by another non-polar amino acid, or a polar amino acid by another polar amino acid, in particular replacement of one amino acid from the group consisting of Ala, Val, Ile, Leu and Met by another amino acid of the same group, replacement of Asp by Glu and vice versa, replacement of Asp by Asn and vice versa, replacement of Glu by Gln and vice versa, replacement of Asn by Gln and vice versa, replacement of one amino acid from the group consisting of Lys, Arg and His by another amino acid of the same group, replacement of one amino acid from the group consisting of His, Lys, Arg, Asp and Glu by another amino acid of the same group, replacement of one amino acid from the group consisting of Phe, Tyr, Trp and His by another amino acid of the same group, replacement of one amino acid from the group Tyr, Thr and Ser by another amino acid of the same group, or replacement of Phe by Tyr or vice versa.

Such variants may be naturally occurring in Mimivirus or in other related virus sources, but may be preferably formed by mutation techniques known in the art selecting for improved (more effective or more selective) hydroxylase properties. Variants may preferably be produced using protein engineering techniques known to the skilled person and/or using molecular evolution to generate and select proteins with improved prolyl hydroxylase and lysyl hydroxylase properties, respectively. Such techniques are e.g. site directed mutagenesis, saturation mutagenesis, error prone PCR to introduce variations anywhere in the sequence, and DNA shuffling used after saturation mutagenesis. With the aid of phage display methods or enzymatic assays of varying designs, mutants may be selected with significantly increased hydroxylase activity or significantly increased selectivity for proline or lysine or substrates in general. Additionally, mutants may be identified through various in vivo mutagenesis strategies which may or may not include high-throughput screening of cells containing the desired mutant activity using, as an example, fluorescence assisted cell sorting of cells containing a fluorescently labelled enzyme substrate as the readout. Another in vivo mutagenesis strategy would involve the addition of various mutagenic compounds to cells containing a plasmid with the gene of interest and use of an appropriate screening method to identify cells harbouring mutants with the desired change in activity or specificity.

More particularly, the invention relates to an isolated protein comprising the sequence of SEQ ID NO:1, of SEQ ID NO:2, or of SEQ ID NO:3. Most preferred is an isolated protein of the sequence SEQ ID NO:1, SEQ ID NO:2 or SEQ ID NO:3, preferably SEQ ID NO:1 or SEQ ID NO:3. As will be shown below, the shorter version of the prolyl hydroxylase proved to be more efficient in E. coli.

In another aspect, the invention relates to an isolated nucleic acid coding for the mentioned proteins, e.g. for a preferred protein.

In particular, the invention relates to an isolated DNA comprising a DNA of the sequence SEQ ID NO:4; SEQ ID NO:5; SEQ ID NO:6; and variants of such DNA comprising variants of SEQ ID NO:4, SEQ ID NO:5 or SEQ ID NO:6 in which one or more, in particular between one and ten nucleotides, are replaced by other nucleotides in a triplet codon coding for the same amino acid as the original triplet codon, and/or one, two, three, four, or five, triplet codons are replaced by triplet codons coding for a different amino acid. More specifically the invention relates to an isolated DNA as defined for use in expressing a protein with hydroxylase properties in a host cell.

Triplet codons are universal for any host cell. Almost all amino acids are encoded by more than one triplet codon. Replacing a nucleotide in a triplet codon by another nucleotide may therefore provide the same amino acid on expression of the DNA. To some extent efficiency of expression of a triplet codon in a host cell is determined by the particular nucleotides in the triplet codon. Replacing nucleotides in triplet codons may increase availability of the same amino acid (and hence expression of the protein) in a particular host cell.

Substituting triplet codons in a DNA of the invention by other triplet codons coding for a different amino acid is also considered part of the invention. One, two, three, four, or five triplet codons may be replaced by other triplet codons, giving rise to a protein variant according to the invention with corresponding number of substituted amino acids on expression. The replacement triplet codons are preferably chosen such that the amino acid substitutions indicated as preferred in the description above are obtained on expression of the DNA.

Methods for exchanging nucleotides in polynucleotides are well known in the art. For example, site directed mutagenesis, saturation mutagenesis, error prone PCR to introduce variations anywhere in the sequence, and DNA shuffling used after saturation mutagenesis may be used to improve protein stability or enzymatic activity of the encoded protein.

In another aspect, the invention relates to a vector comprising a DNA as defined above, in particular a bicistronic vector comprising a DNA of the sequence SEQ ID NO:4 and of the sequence SEQ ID NO:6, and variants thereof as defined above.

Vectors considered are those suitable for expression in a particular host cell, in particular those mentioned below as being preferred, and are well known in the art. Particular vectors considered are pET-series vectors from Novagen, such as pET16b, pET22b, pET28a/c, pET32a/b/c, pETcoco-1, and pETM-50, featuring alternative antibiotics resistance, e.g. resistance to ampicillin, kanamycin, chloramphenicol, or tetracycline. Other possible expression vectors are the pFN18, pFN19, pFN20, or the PinPoint-Xa series of vectors from Promega, the pMAL-c5, pMAL-p5, pMAL-c5E, pMAL-p5E, pMAL-c5G, and pMAL-p5G series of vectors from New England Biolabs, the pFlag, pTac, and pT7 vector series from Sigma, pBAD/His, pEM7/Zeo, pRSET, pTrcHis, pTrcHis2 from Invitrogen, pCAL-c, pCAL-n, pCAL-n-ek, pCAL-n-FLAG, pBEn-SBP, pBEn-SBP-SET1, pBEn-SBP-SET2, pBEn-SBP-SET3, pBEn-SET1, pBEn-SET2, or pBEn-SET3 from Agilent, pHAT10, pHAT11, pHAT12, or pHAT20 from Clontech, or the pQE series of vectors from Qiagen.

For simultaneous expression of a prolyl hydroxylase of the invention and a lysyl hydroxylase of the invention in a particular host cell, a bicistronic vector is preferred. Such bicistronic vectors are, for example, pET-series vectors engineered to include two T7 promoter-controlled cassettes or the pRSFDuet-1 vector from Novagen. Bicistronic bacterial expression vectors can be easily engineered by duplicating an expression cassette in any plasmid, in particular those plasmids mentioned above.

In yet another aspect the invention relates to host cells comprising a vector of the invention. Preferred host cells are those known to be suitable for large scale expression of exogenous proteins, for example E. coli BL21(DE3), BL21(DE3)-pLysS, BL21(DE3)-pLysE, BL21-SI, BL21-AI, Rosetta, Rosetta-pLysS, HMS174, BLR, CD41(DE3) and CD43(DE3). However, other host cells are likewise considered, for example archaea, gram-positive bacteria, yeast, fungi, plant, and animal cells, in particular insect cells.

Most preferred host cell is E. coli, e.g. E. coli BL21(DE3).

In a particular embodiment, the invention relates to a host cell, such as E. coli, expressing collagen or proteins containing collagen domains, a prolyl hydroxylase of the invention, and a lysyl hydroxylase of the invention, preferably collagen, a protein comprising the sequence SEQ ID NO:1 and a protein comprising the sequence SEQ ID NO:3.

The invention further relates to methods of expressing a hydroxylase comprising culturing a host cell comprising a vector of the invention. The target proteins can be expressed in the cytoplasm or periplasm of bacteria or in animal cells using dedicated expression vectors such as recombinant baculoviruses in Autographa californica insect cells.

In a further aspect the invention relates to the synthesis of collagen and proteins comprising collagen domains in a host cell, for example in a bacterial host cell such as E. coli, comprising culturing a host cell comprising DNA coding for collagen or for a protein with a collagen domain and for a hydroxylase of the invention. The target proteins, including collagen and hydroxylases, can be expressed in the cytoplasm or periplasm of E. coli using dedicated signal peptides. After lysis of the cells, collagen can be isolated by affinity chromatography and/or selective precipitation using NaCl. Collagen can be expressed as full-length procollagen protein, that is including propeptide fragments, or as truncated proteins including telopeptides but devoid of propeptide fragments. When expressed as procollagen, propeptide fragments can be removed by pepsin digestion, which leaves the triple helical domain intact. When collagen is expressed without propeptides, pepsin digestion can be omitted, which also leaves telopeptide sequences intact and thereby allows formation of collagen fibrils at a later stage.

Collagen considered is, in particular human collagen, such as collagen III. In addition to the family of true collagens, many collagen domain containing proteins such as adiponectin, complement Clq subunit a, b, and c, complement Clq-like protein 2, 3, and 4, Clq-related factor, complement Clq tumor necrosis factor-related 1, 2, 3, 4, 5, 6, 7, 8, 9, and 9B, collagen and calcium-binding EGF domain-containing protein 1, collectin 10, 11, and 12, acetylcholinesterase collagenic tail peptide, collagen triple helix repeat-containing protein 1, ectodysplasin A, EMI domain-containing protein 1, elastin microfibril interface located protein 1 and 2, ficolin 1, 2, and 3, gliomedin, macrophage receptor MARCO, mannose-binding protein C, macrophage scavenger receptor types I and II, neurogranin, otolin-1, scavenger receptor class A number 3 and 5, pulmonary surfactant-associated protein A1, A2, and D, and WD repeat-containing protein 33 can be manufactured by the method of invention in a suitable host cell, for example bacteria, such as E. coli.

There are 29 known human collagens, composed of proteins from at least 46 different genes (Shoulders, M. D. and Raines, R. T. (2009) Annu Rev Biochem, 929-58; Schegg, B., Hülsmeier, A. J., Rutschmann, C., Maag, C., and Hennet, T. (2009) Mol Cell Biol 29, 943-952). Any of these 46 genes in addition to any other useful animal collagens could be inserted into this system to produce the desired collagen product. Since many collagens are heterologous in nature, production of any type of collagen could be achieved by expression of the appropriate combination of collagen subunit genes for a given type of collagen in this system. Alternatively, each collagen subunit could be produced separately and then the final collagen product assembled in vitro by mixing the components in appropriate ratios with the necessary enzymes. These could include lysyl oxidases, hydroxylysine glycosyltransferases such as GLT25D1 and GLT25D2, proteases for the removal of the amino and carboxy-terminal propeptides, and enzymes involved in disulphide bond formation and shuffling such as protein disulphide isomerase and thioredoxin.

Furthermore the invention relates to recombinant collagen and recombinant proteins comprising collagen domains, incorporating hydroxylated prolines and hydroxylated lysines, and manufactured in a bacterial host such as E. coli. These collagens and proteins comprising collagen domains have useful applications, as is described below.

The invention relates to the mentioned hydroxylases for use in the hydroxylation of collagen, in particular for in situ hydroxylation of collagen in a host cell. Ample experimental proof is provided that expression of the hydroxylases of the invention in the presence of collagen leads to collagen containing hydroxylated proline and/or hydroxylated lysine.

Likewise the invention relates to the mentioned lysyl hydroxylases for the glycosylation of hydroxylysine in collagen and hydroxylysine containing proteins, in particular for in situ glycosylation of collagen and hydroxylysine containing proteins in a host cell. Hydroxylation of lysine is the first step in glycosylation of lysine. The vertebrate collagen peptide O-galactosyltransferases GLT25D1 and GLT25D2, as well as a related protein and potential collagen O-galactosyltransferase CEECAM1 have recently been identified and are available to initiate glycosylation on the E. coli produced hydroxylysine containing proteins (Schegg, B., Hülsmeier, A. J., Rutschmann, C., Maag, C., and Hennet, T. (2009) Mol Cell Biol 29, 943-952). Additionally, any other hydroxylysine glycosyltransferases can be used to further modify hydroxylysine containing proteins produced in this system. The glycosylation proceeds in vivo by addition of the necessary glycosyltransferases to the E. coli expression system using dedicated expression vectors, or in vitro using purified or partially purified enzyme and the hydroxylated protein produced according to the invention.

The invention further relates to the use of the described hydroxylases and nucleotides encoding these in a gene therapy setting, including, but not limited to epigenetic modulation through modification of various factors. The hydroxylases of the invention and nucleotides encoding these may be applied in a gene therapy setting to alter the prolyl and lysyl hydroxylation pattern of endogenously produced collagen or of proteins containing collagen domains. Various forms of osteogenesis imperfecta or of the Ehlers Danlos syndrome are candidate for such an approach. The hydroxylases and nucleotides encoding these can be introduced into tissues of interest using a lentivirus or other form of vector, allowing the enzymes to substitute for the defect in the target cells.

The invention further relates to the use of the described hydroxylases in a method of achieving expression of proteins with collagen domains or prolines and lysines available for modification. It is not uncommon for a recombinant protein of interest to fail to express in bacteria or other cell culture. In the case of post-translationally modified proteins, if the expression system does not contain the appropriate post-translational modification machinery, recombinant expression is often particularly problematic. Many modifications affect protein folding and solubility, such that even if the protein of interest is expressed, it may not be in a soluble or functional form. The hydroxylases of the invention and nucleotides coding for it, while intended first and foremost to produce collagen products for clinical use, may also be used for biotechnology purposes. Should one wish to recombinantly express a protein with a collagen domain, or a protein with prolines or lysines available for hydroxylation, the system of the invention can be applied for production of properly folded and functional protein. This may result in increased overall production, or may improve the quality or functionality of the protein produced.

The invention further relates to the use of the described hydroxylases in the production of proteins with hydroxylated amino acids and glycosylated hydroxy-amino acids including applications such as protein arrays. Production of arrays with various collagens, collagen components, or proteins with collagen domains, or proteins with prolines and lysines available for hydroxylation can benefit from the presence of the post-translational modifications provided by the system of the invention. Proteins containing glycosylated hydroxy-amino acids can be produced with the addition of the appropriate enzyme and substrate components either in vivo or in vitro.

The invention further relates to the use of the described hydroxylases in the production of vaccine components or antibodies, e.g. for research, biotechnology, or clinical use. The hydroxylases of the invention are capable of producing proteins containing only the post-translational modifications required on the potential antigenic structures. This enables the production, as one example, of antibodies to specific sites in a specific collagen or collagen chain, capable of discerning whether a given amino acid is post-translationally modified or not. Antigenic peptides of interest can be hydroxylated on selected lysine and proline residues in vitro. These hydroxylated peptides are purified by gel filtration and used alone or in combination with adjuvants to stimulate immune cells in vitro or to elicit an immune response in vivo. The availability of hydroxylated peptides allows the production of glycosylated collagen peptides after reaction with the GLT25D1 collagen galactosyltransferase in vitro. Glycosylated peptides can be purified by gel filtration and used in immunological applications.

The invention further relates to the use of the described hydroxylases in the production of other enzymatically modified compounds. For example, tRNA molecules charged with either proline or lysine can be hydroxylated by the enzymes of the invention. Additionally, these tRNA molecules possessing hydroxylated proline or lysine can be further modified by glycosylation. This approach allows the use of non-natural charged tRNA molecules either in vivo or in vitro during the translation process to incorporate specific functionalities in recombinant peptides and proteins.

The invention further relates to the use of collagen products and proteins comprising collagen domains manufactured by the method of the invention in a clinical setting and as a replacement of animal collagen-based products applied in wound healing and surgery. The production of recombinant human collagen in bacteria enables controlling the degree of hydroxylation, thereby matching the need for different biophysical properties in different tissues settings. The application of bacterially produced recombinant human collagen avoids potential antigenic reactions associated with the use of animal products and the possibility of contamination with potentially infectious agents such as viruses and prions.

To enable collagen production, a DNA encoding a given collagen subunit is sub-cloned into a bacterial expression vector. Signal sequence tags enabling the targeting of the recombinant protein to specific cellular compartments, and tags for the purification of recombinant collagen are added to the N-terminal end of the collagen cDNA. Examples of such tags are His₆₋₁₀, streptavidin binding peptide, galectin-1, calmodulin binding peptide, biotin, FLAG, or histidine affinity tag (HAT). Intervening sequences containing protease cleavage sites allowing for release of the tag from the protein of interest are also added to the cDNA construct. Proteases preferably used in this fashion are tobacco etch virus (TEV) protease, thrombin, factor Xa, genenase I, and enterokinase. Signal sequences designed to elicit secretion into the periplasmic space (such as a pelB leader sequence in pET22 as one example) can be included in the expression construct. These tags, protease cleavage sites, and leader sequences may be contained in various expression vectors allowing a simple sub-cloning of the gene of interest from the cDNA to the vector, or may be introduced into any chosen vector by inclusion of the tags with the gene of interest during sub-cloning into vectors allowing expression of the protein of interest in the cytosol of the host cell such as E. coli or secretion into the periplasmic space.

Purification of recombinant collagen begins with release of the protein by an osmotic shock in the case of periplasmic expression. Purification of protein expressed in the cytosol first requires lysis of the bacteria, either through freeze-thaw cycles, french press, sonication, the use of lysozyme, various commercial products such as FastBreak Cell Lysis Reagent (Promega), or any combination of these techniques. Protein expressed in the cytosol may be in a soluble state and suitable for immediate purification, or may be present as insoluble matter or inclusion bodies in which case a solubilization step is first necessary prior to purification.

Once the protein of interest is available in a soluble form, purification proceeds utilizing the purification tag attached to the protein. For example, in the case of His₆₋₁₀ tags, this entails purification of the protein by affinity chromatography over a nickel bead column or in a batch purification using a free nickel bead matrix. In the case of affinity purification by chromatography, the protein solution is injected over a previously equilibrated column containing nickel beads allowing the tagged protein to bind. The protein is then washed with various buffers to remove contaminating proteins, and is then eluted with a gradient of imidazole. A batch purification protocol differs only in that the beads must be incubated with the protein of interest allowing the tagged protein to bind to them, at which point the beads may be poured into a column for washing and elution steps, or the washing and elution steps can be performed by successive pelleting of the beads in a centrifuge and removal of the supernatant. Purifications utilizing other purification tags are similar, differing only in the matrix that the proteins bind to, and the compounds necessary to elute the protein from a given matrix. After purification, recombinant collagen is renatured in 0.2-0.4 M NaCl, 0.1 M TrisHCl buffer, pH 7.4 by heating at 45° C. for 20 min followed by rapid cooling to 20° C. and incubation at 20° C. for 2-8 h. For heterologous collagens, such as collagen types I, IV, and V, the appropriate ratio of collagen strands first needs to be mixed in vitro after purification, or the appropriate components needs to be expressed together using a bi- or tricistronic vector in vivo. The resulting triple-helical collagen is resistant to pepsin digestion (1.5 mg/ml in 10 mM acetic acid) while residual co-purified proteins are degraded by this enzyme.

Removal of purification or solubility enhancement tags can be achieved after elution from the chosen purification matrix with the appropriate protease. Alternatively, this may be achieved by on column digestion with the enzymes, allowing the purified protein to elute from the column while the tags remain bound to the purification matrix. The small amount of contaminating enzyme remaining after this step can be removed by polishing steps using gel filtration or ion exchange chromatography, or in some cases, by affinity chromatography due to the presence of an affinity tag engineered on the protease. The protein of interest is washed through the affinity matrix without binding while contaminants are retained on the matrix.

After purification of triple-helix collagen, higher order structures such as homotypic and heterotypic fibrils can be produced in vitro. First, propeptides are removed by protease treatment as described above if the recombinant collagen produced includes such propeptides. Alternatively, recombinant collagen lacking propetide sequences can be produced. In such cases, protease treatment is avoided to preserve telopetides, which are relevant to fibril formation. Depending on the higher order structure, different recombinant collagens are mixed at various ratios and incubated with recombinant lysyl oxidase enzyme to catalyse the formation of covalent cross-links between collagen strands and helices. After this step, collagen fibrils are collected by gel filtration.

Many collagens are heterotypic, thereby including small amounts of other types of collagen in addition to a main type of collagen. Type I collagen for example has been reported to contain small amounts of type III, V, and XII. The present system allows production of any type of collagen protein, which can be mixed in the appropriate ratios during production of higher order collagen structures. This system makes possible extreme fine tuning of the collagen production process so that the product can resemble the natural collagen as closely as possible.

The invention further relates to the use of collagen and proteins comprising collagen domains as scaffold for tissue or organ growth in vitro and in vivo. The ability to rejuvenate and regrow organs to replace failing ones relies on extracellular matrices based on collagen. Non-cross-linked collagen according to the invention can be used in combination with hydroxyapatite to produce tooth and bone fillers or coating implants. Recombinant hydroxylated collagen produced with the present invention can be applied in vitro on prosthetic implants and fixed by cross-linking using lysyl oxidase enzymes. In the case of tooth and bone replacement, collagen fibrils produced as described above are incubated on a polyurethane or ceramic scaffold together with 50-80% hydroxyapatite (% total weight) at pH 8 first incubated at 40° C. for 30 min, then 30-35° C. for up to 50 days. Coatings for prosthetic implants can similarly be produced by providing collagen fibrils and hydroxyapatite and cross-linking by UV light. In this fashion, prosthetic joints can be covered in cartilage-like coatings, or an appropriate collagen like-matrix which serves as a lattice for the patient's own body to recreate the natural collagen based tissues in the joint. Fibrils of recombinant collagen can also be used as matrix to support the tridimensional growth of cells. These cells will adhere to the collagen mimicking the natural extracellular matrix they would find themselves in during development, creating a suitable environment for them to differentiate and replicate.

The invention further relates to the use of collagen and proteins comprising collagen domains as an additive in cell culture for improved cell viability and adhesion. Cells typically grow in contact with other cells and with the extracellular matrix. The composition of the extracellular matrix is known to affect the viability, growth characteristics, and morphological characteristics of various cultured cells. The ability to produce cell culture plates or three-dimensional matrices including various types of collagens can be used to improve cell culture methods for various cells.

The invention further relates to the use of collagen and proteins comprising collagen domains for the purification or identification of molecules or cells that interact with collagen. Collagen contributes to the selective isolation of specific cell types or cell fragments like platelets from serum simply by culturing on plates coated with collagen and allowing cells that specifically bind to collagen to interact with the collagen coating. The same approach can be used to isolate proteins and small molecules from biological fluids based on their interaction with collagen.

The invention further relates to the use of collagen and proteins comprising collagen domains as an assay substrate in either a research, clinical, or industrial setting: Recombinant hydroxylated collagen is useful in a laboratory setting as a standard assay substrate for collagen modifying proteins and for collagen-specific proteases. Collagen produced under defined and specific quality is preferable over animal collagen.

The scientific rationale for the invention is now described in more detail, without limiting the invention to the particular proteins, polynucleotides, vectors and host cells described.

The genome of the giant virus Mimivirus encodes eight collagen proteins of unknown function. By searching through the Mimivirus genome for genes structurally similar to known collagen hydroxylase genes, the two open reading frames L593 and L230 could be retrieved. The open reading frame L593 was similar to prolyl hydroxylase genes found in plant and animal cells, whereas the open reading frame L230 was similar to several animal lysyl hydroxylase genes. Closer examination of the open reading frame L593 revealed two possible ATG initiation codons, which could yield proteins of 242 or 222 amino acids. The long form of the open reading frame L593 is 729 bp long and is terminated by a TAA stop codon (SEQ ID NO:5).

Like most genes of the Mimivirus genome, the long form of the open reading frame L593 is AT-rich (71.3%). The short form of the open reading frame L593 is 669 bp long shares the same termination codon as the long form but starts at an ATG codon 69 nucleotides downstream of the first one (SEQ ID NO:4).

The open reading frame L230 is 2688 bp long (SEQ ID NO:6) and encodes only one possible protein of 895 amino acids. The open reading frame L230 is also AT-rich with a content of 68.8% AT nucleotides.

The predicted sizes of the L593 long and short isoforms are 242 (SEQ ID NO:2) and 222 (SEQ ID NO:1) amino acids long, which result in proteins of 30 kDa and 28 kDa, respectively.

The short and long forms of the L593 protein share 91% sequence identity and when compared to prolyl hydroxylases from various genomes, similarities ranging between 50% and 70% could be identified with bacterial and plant prolyl 4-hydroxylase proteins. The sequences similar to animal prolyl 4-hydroxylases were limited to short motifs and the overall homology was only around 15%. The L230 protein has a predicted size of 895 amino acids weighing 103 kDa (SEQ ID NO:3).

The L230 protein shares sequence similarity with animal lysyl hydroxylases in a range between 40 to 60%. The similarity is highest in the second half of the L230 protein, where several sequence motifs known to be important to the lysyl hydroxylase activity can be identified.

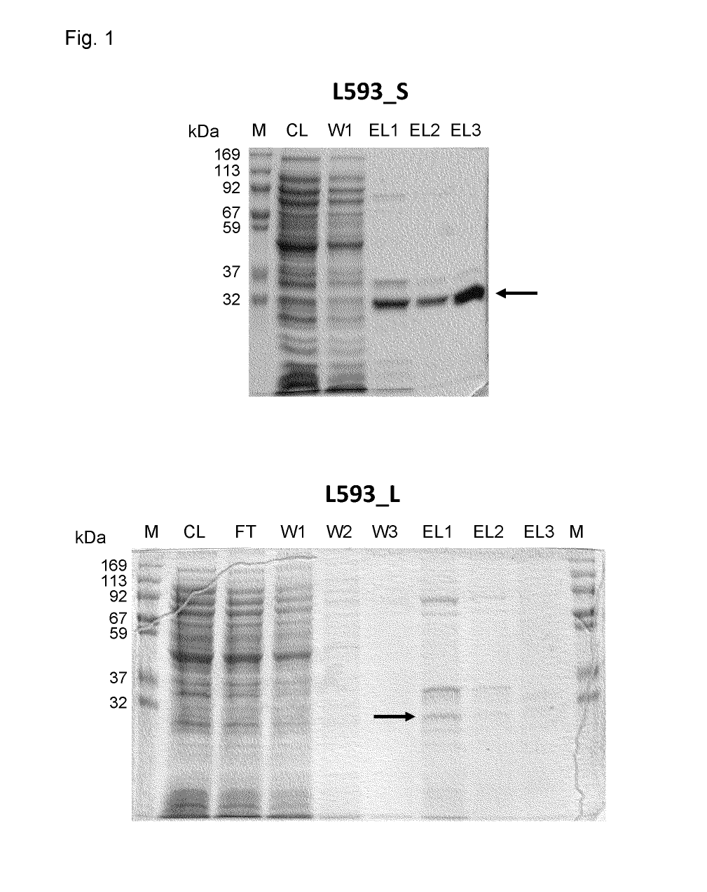

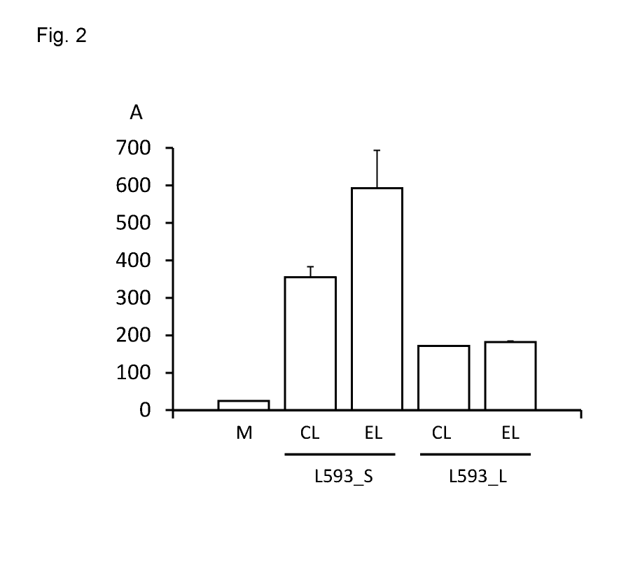

To determine the stability and the enzymatic activity of the two L593 encoded protein isoforms, the short and the long forms of the open reading frame L593 were expressed using the pET16b expression vector in BL21(DE3) E. coli. A His₁₀-tag was added at the N-terminus of the proteins to allow their purification by affinity chromatography on Ni-sepharose columns. The expression of both L593 isoforms was not toxic to E. coli cells as the yield of transformants and the growth rate of the resulting clones was similar to those of mock-transformed E. coli cells. The short form of the L593 protein was produced at high levels, achieving a yield of at least 10 mg protein per liter of culture, and could be purified to near homogeneity from E. coli (FIG. 1). By contrast, the long form of the L593 protein was produced at a lower yield and appeared less stable as evidenced by the possible cleaved fragments co-eluting during the purification procedure (FIG. 2).

The prolyl hydroxylase activity of the recombinant short and long forms of L593 was assayed by measuring [¹⁴C]-CO₂ production from [¹⁴C]-2-oxoglutarate as described previously (Kivirikko, K. I., and Myllyla, R. (1982) Methods Enzymol 82 Pt A, 245-304). The prolyl hydroxylase activity was tested in the lysate of E. coli expressing the L593 constructs and in semi-purified fractions after affinity chromatography. Both short and long forms of L593 are active as prolyl hydroxylases, but the short form has a higher specific activity than the long form (FIG. 2). The prolyl hydroxylase activity of the long form was not increased after affinity chromatography, indicating that this isoform is possibly unstable when purified. Since the short form of L593 yielded prolyl hydroxylase activity in the range of 600 pmol/min/mg protein, this isoform was used for subsequent experiments.

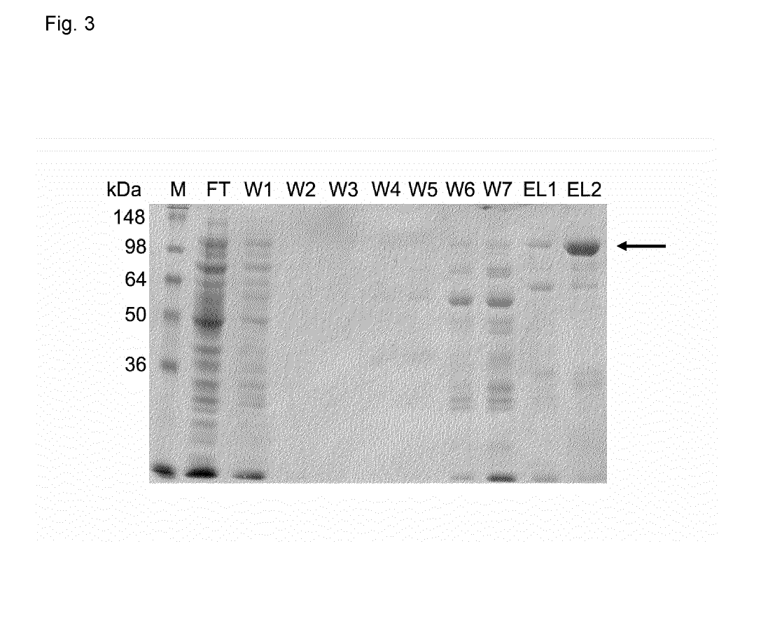

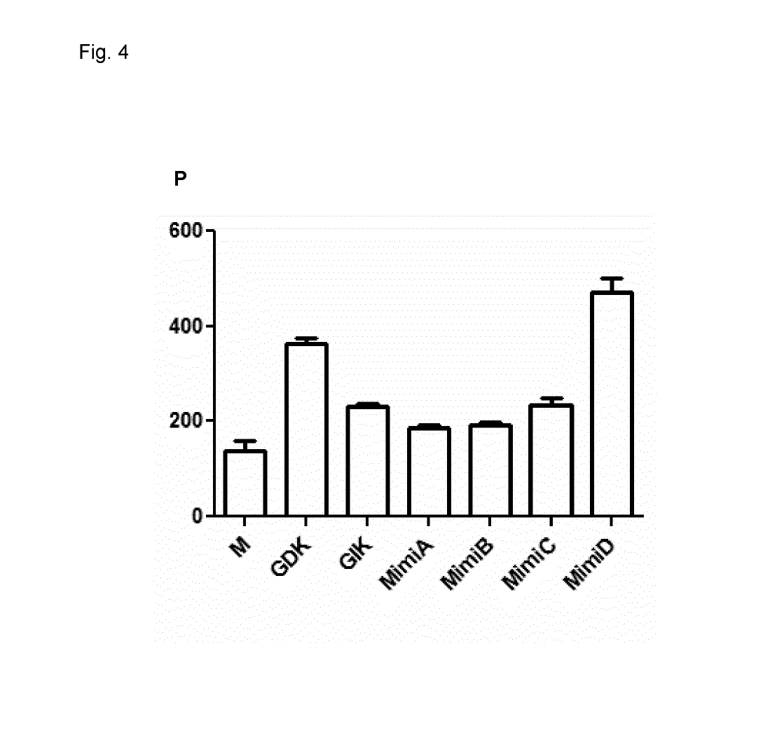

The L230 construct was cloned into the pET16b expression vector and transformed into BL21(DE3) E. coli as done for the L593 protein. As observed for the L593 protein, the expression of L230 does not impair the viability and growth rate of E. coli. The L230 protein was purified by affinity chromatography using Ni-sepharose, which yielded the expected protein of 103 kDa at near homogeneity (FIG. 3). The purified L230 protein was stable for 1 week when stored at 4° C. and for at least 1 year after freezing. The lysyl hydroxylase activity of the purified L230 protein was assayed on several peptide acceptor substrates. This experiment confirmed the identity of L230 as a lysyl hydroxylase enzyme. The lysyl hydroxylase activity was highest towards the (GDK)₄ peptide and the GTKGETGLKGII peptide derived from a Mimivirus collagen sequence (FIG. 4).

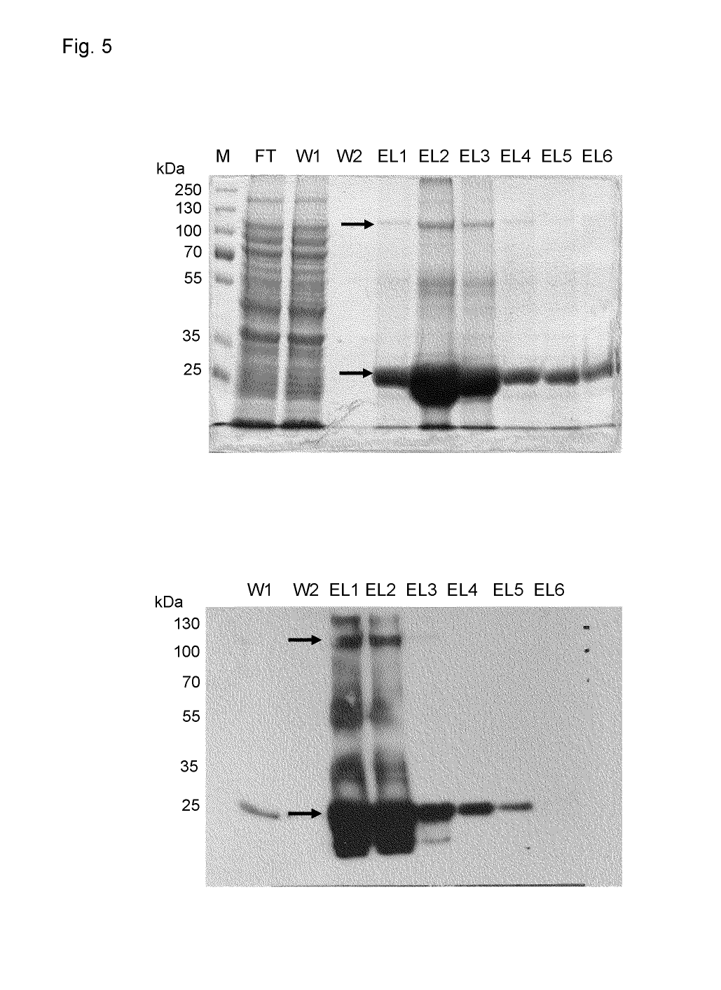

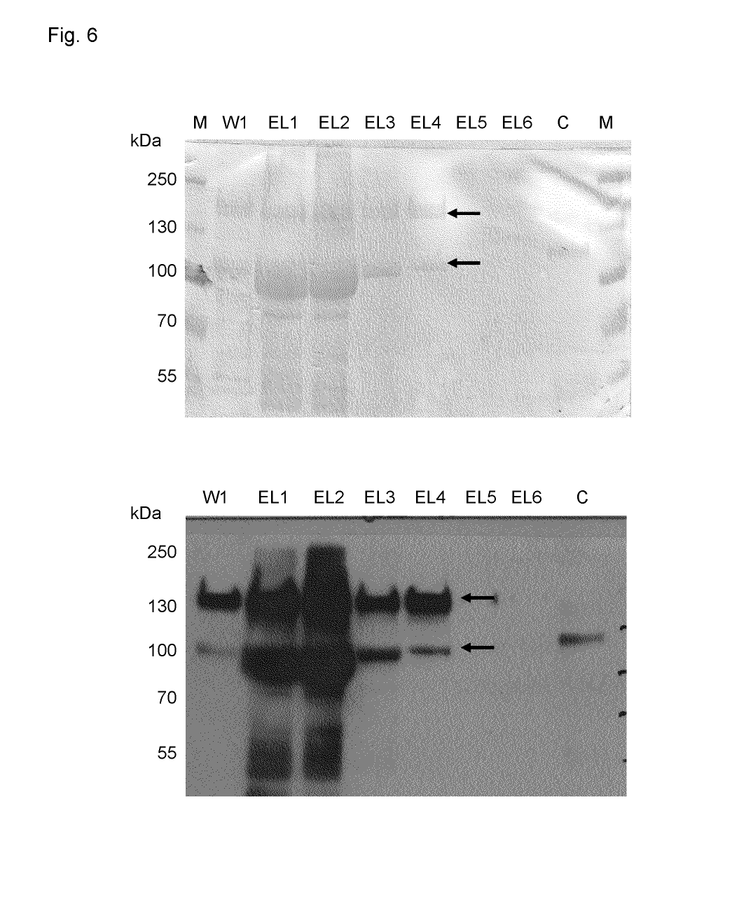

Since both Mimivirus L593 and L230 proteins were confirmed as being active prolyl and lysyl hydroxylase enzymes, respectively, the co-expression of these proteins with a His₁₀-tagged human collagen III construct in E. coli was investigated. The combined expression of the Mimivirus L539 prolyl hydroxylase and human collagen III was well tolerated in E. coli as no change in viability and growth rate could be detected. Both L593 and human collagen III proteins were expressed in E. coli and could be purified by Ni-sepharose affinity chromatography (FIG. 5). The human collagen III protein migrated faster in polyacrylamide gels than according to its predicted molecular weight of 86 kDa for the human collagen III construct used. As expected, the small L593 protein was expressed at higher levels than the bulky collagen III protein (FIG. 5). The co-expression of Mimivirus L230 lysyl hydroxylase and human collagen III in E. coli was also investigated. As observed for the co-expression with L593, both collagen III and L230 proteins were efficiently produced in E. coli without any sign of toxicity. The two His₁₀-tagged proteins could be purified by Ni-sepharose affinity chromatography without degradation and without loss of enzymatic activity for L230 (FIG. 6).

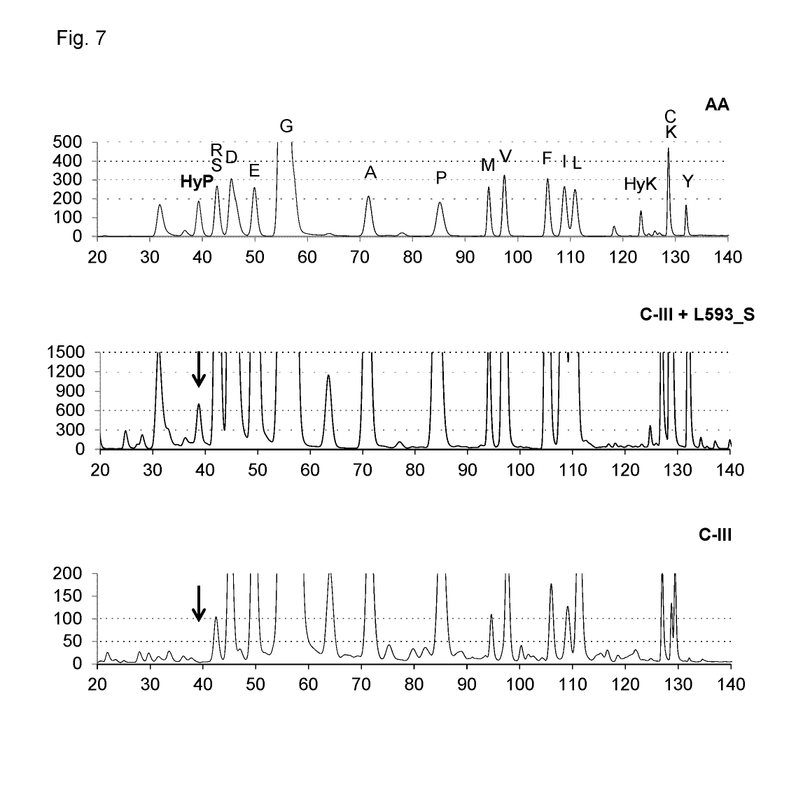

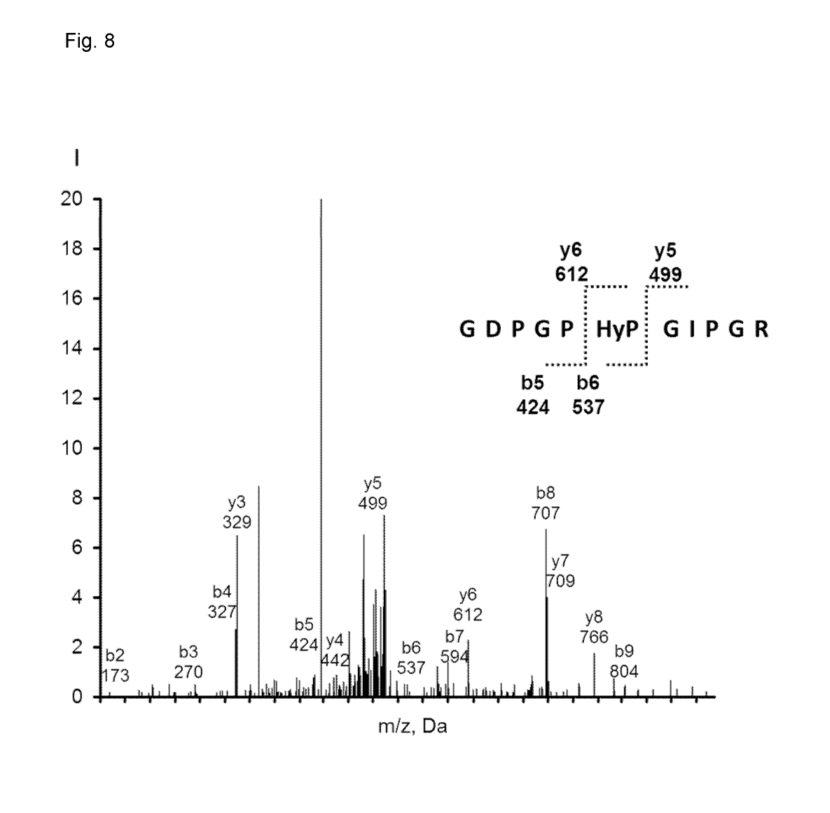

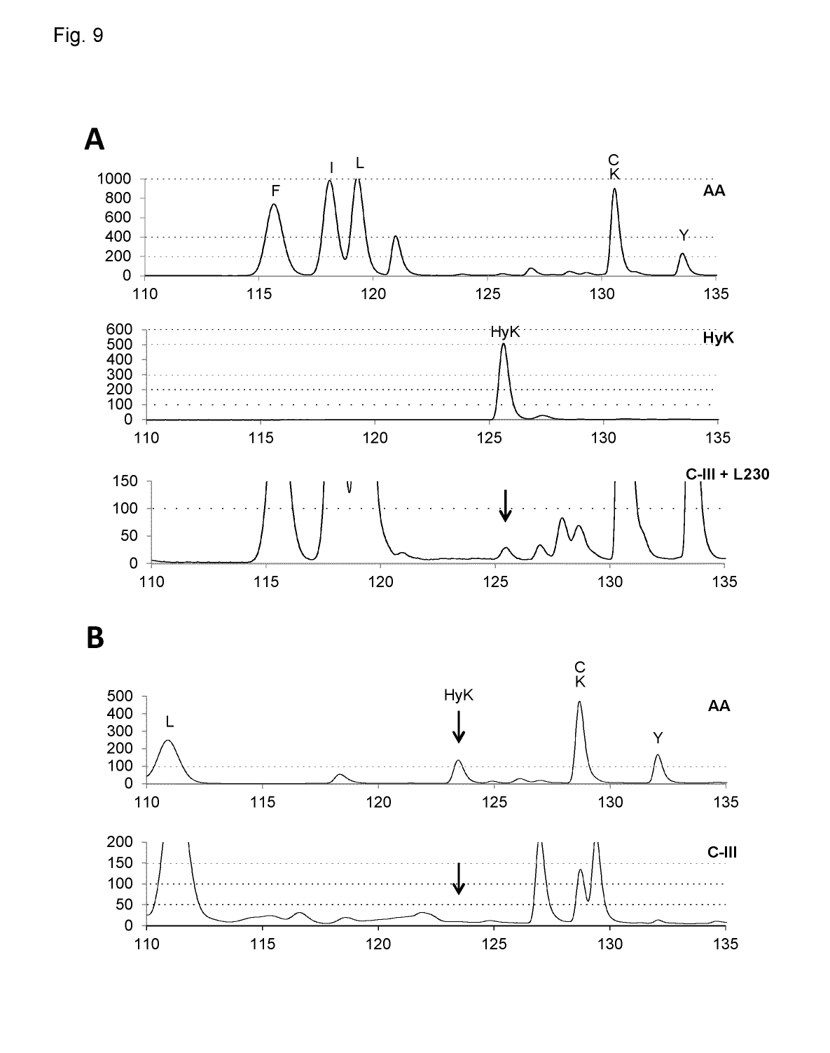

To demonstrate that the Mimivirus L593 and L230 hydroxylases are able to hydroxylate collagen in vivo in E. coli, the amino acid composition of the human collagen III protein co-expressed with either the L593 prolyl hydroxylase or the L230 lysyl hydroxylase was analyzed. The collagen III construct was purified by affinity chromatography and hydrolyzed in 6 M HCl for 24-48 h. The resulting amino acids were fluorescently labeled with FMOC and separated by HPLC (Hitachi, Merck). Hydroxyproline could not be detected in the human collagen III construct expressed alone in E. coli (FIG. 7). By contrast, the amino acid hydroxyproline was clearly detected in the human collagen III extract when the Mimivirus L593 protein was co-expressed (FIG. 7). To prove that hydroxyproline was present in the collagen III protein, the purified collagen III construct was fragmented with trypsin and the resulting peptides subjected to tandem mass spectrometric analysis. Several peptides contained hydroxyproline in the collagen motif G-X-Y (FIG. 8). This finding confirmed the prolyl hydroxylase activity of the Mimivirus L593 protein towards human collagen III produced in E. coli. The co-expression of Mimivirus L230 lysyl hydroxylase and human collagen III in E. coli also led to the in vivo hydroxylation of lysine residues as demonstrated by amino acid analysis. A peak corresponding to hydroxylysine was detected in collagen III co-expressed with L230 but not in collagen expressed alone in E. coli (FIG. 9).

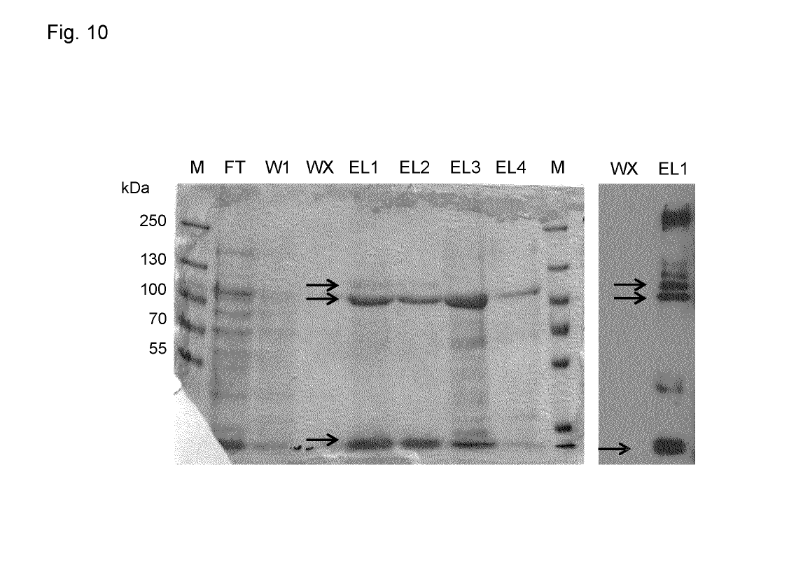

After having shown that a human collagen III construct could be hydroxylated at proline and lysine residues when co-expressed with the corresponding Mimivirus L593 and L230 hydroxylases, a bicistronic expression vector enabling the dual expression of both L593 and L230 genes in E. coli was built. The transformation of E. coli with the expression vector encoding human collagen III and the bicistronic vector encoding L593 and L230 hydroxylases was well tolerated and yielded the three recombinant proteins in the milligram range per liter of bacterial culture. The three proteins could be purified by affinity chromatography to near homogeneity (FIG. 10). The amino acid analysis of purified collagen III showed the presence of hydroxyproline and hydroxylysine residues, thereby demonstrating the effectiveness of the Mimivirus hydroxylases expressed in E. coli.

The simplicity of the expression system and the convenience of the bicistronic vector allow the efficient post-translational hydroxylation of proteins containing collagen domains. In addition to the family of true collagens, several collagenous proteins like the hormone adiponectin, the immune protein mannose-binding lectin and the surfactant proteins A and D can be produced at high yield in bacteria.

### EXAMPLES

### Cloning of Expression Vectors

L593_short and L593_long were cloned as a XhoI-BamHI PCR fragment into pET16b using the primers

5′-TGACCTCGAGGTATTGTCAAAATCTTGTGTGT-3′ (SEQ ID NO:14) and

5′-CAGGGATCCATTTTGTGTTAAAAAAATTTTAGG-3′ (SEQ ID NO:15) (L593_short),

5′-TGACCTCGAGAAAACTGTGACTATCATTACAATA-3′ (SEQ ID NO:16) and

5′-CAGGGATCCATTTTGTGTTAAAAAAATTTTAGG-3′ (SEQ ID NO:15) (L593_long). The full-length human collagen III COL3A1 cDNA was cloned by PCR using the primers

5′-GGCAAGCTTTGGTGAGCTTTGTGCAAAAGG-3′ (SEQ ID NO:17) and

5′-ATCGCGGCCGCTTATAAAAAGCAAACAGGGCCAACGTC-3′ (SEQ ID NO:18) as a HindIII-NotI fragment into pET28a. A shorter version of human COL3A1 was generated by excision of an internal Alel-Mscl fragment (1191 bp). The bicistronic L230/P4H_short vector was prepared by inserting the expression cassette from the pET16b-P4H_short as a BglII-HindIII fragment into the pET16b-L230 vector opened with BamHI-HindIII. The pET16b-L230 expression vector was created by first isolating Mimivirus genomic DNA according to Raoult et al. (2004) Science 306, 1344-1350. The L230 gene was amplified from the genomic DNA by PCR with the primers

5′-GACCCATGGGATCCATTAGTAGAACTTATGTAAT-3′ (SEQ ID NO:19) and

5′-GTCACTAGTTTAATTAACAAAAGACACTAAAATAT-3′ (SEQ ID NO:20) (Microsynth). The amplification primers incorporated a 5′ NcoI and a 3′ SpeI restriction endonuclease site respectively which were used to clone the fragment into the plasmid pFastBacI (Invitrogen). The L230 gene was subsequently amplified by PCR using the pFastBac construct as template, and the primers

5′-TGACCTCGAGATTAGTAGAACTTATGTAATT-3′ (SEQ ID NO:21) and

5′-CAGGGATCCGTCCAATAAAGTGTATCAAC-3′ (SEQ ID NO:22) incorporating a 5′ XhoI site and a 3′ BamHI site into the amplicon. The XhoI/BamHI digested amplicon was then ligated into XhoI/BamHI digested pET16b (Novagen) vector.

### Bacterial Expression

The pET16b- and pET28-based expression vectors were transformed into chemically competent E. coli BL21(DE3) using a heat shock and a 1 h recovery step in 1 mL of antibiotic free Luria-Bertani (LB) medium with shaking at 220 rpm at 37° C. The next day, a fresh colony was inoculated into 50 mL of LB medium supplemented with 100 μg/mL ampicillin (Sigma) (LBamp⁺) and/or 50 μg/mL kanamycin (Sigma) (LBkana⁺) and incubated overnight at 37° C. with shaking at 220 rpm. The next morning, 10 mL of the overnight culture was used to inoculate a 1 L culture of LBamp⁺ and/or LBkana⁺ which was incubated at 37° C. with shaking at 200 rpm until an Optical Density at 600 nm (OD₆₀₀) of approximately 0.4 was reached, at which point the temperature was lowered to 32° C. When the OD₆₀₀ approached 0.6, protein expression was induced with the addition of isopropylthio-β-D-galactopyranoside (IPTG) to a concentration of 1 mM. The culture was incubated for a further 3 h after which the bacteria were pelleted at 6000×g at 4° C. for 30 min, and then resuspended in 30 mL of ice cold MCAC10 buffer (20 mM Tris-HCl (Biosolve) pH 7.4, 500 mM NaCl (Sigma), 10 mM imidazole (Sigma), 10% v/v glycerol (ERNE surface AG) prior to freezing at −20° C.

### Protein Purification

One 30 mL frozen pellet of transformed E. coli BL21(DE3) was thawed and lysed under ice-cold conditions using an Emulsiflex C5 French-press (Avestin). The lysed bacteria were immediately clarified by centrifugation at 13,000×g in an SS-34 rotor in an RC-6 centrifuge (Thermo-scientific) for 30 min at 4° C. The clarified lysate was incubated on a rotator at 4° C. in the cold room with 1 mL of a 50% bead slurry of Ni Sepharose High Performance affinity beads (GE Healthcare) previously equilibrated in MCAC10 buffer. The beads were pelleted by centrifugation at 2700×g at 4° C. in a Universal 32R bench top centrifuge (Hettich) for 2 min. The beads were transferred to 1.5 mL microcentrifuge tubes (Trefflab) and all subsequent steps with microcentrifuge tubes were performed on ice or at 4° C. in a 5415 R bench-top micro-centrifuge (Eppendorf) at 16,100×g. The beads were washed in 3 to 5 bed volumes of MCAC10 buffer. The protein was then eluted with 5 consecutive bed volumes of MCAC400 buffer (MCAC10 buffer containing 400 mM imidazole rather than 10 mM) which were consolidated into a single fraction. The eluate was immediately concentrated to 1 mL or less, and the buffer exchanged into MCAC10, using Amicon Ultra Centrifugal Filters (Millipore) with a nominal molecular weight cut-off of 10,000 Da, at 4000×g in a swinging bucket rotor in a Heraeus Cryofuge 6000i centrifuge (Thermo-scientific). The protein was stored in MCAC10 buffer at 4° C. in the cold room until needed.

### SDS-Polyacrylamide Gel Electrophoresis, Western Immuno-Blotting

Samples were prepared in 4×SDS-PAGE loading buffer (200 mM Tris-HCl pH 6.8, 400 mM DTT (Fluka), 8% w/v SDS (Sigma), 40% v/v glycerol, 4 mg/mL Bromophenol Blue (Merck)). Ten μL of sample in 4× loading buffer was electrophoresed through a 10% SDS-PAGE gel using the protocol of Laemmli The gel was then stained with Coomassie blue R250 to visualize protein bands. SDS-PAGE performed in preparation for transfer to nitrocellulose for Western blotting utilized samples diluted 1:100 with 1× loading buffer prior to loading on the gel. After completion of electrophoresis, the proteins were transferred from the gel to an Imobilon-NC nitrocellulose membrane (Millipore) utilizing a wet transfer apparatus (BioRad) and running buffer composed of 25 mM Tris base, 20 mM glycine (Fluka), 20% v/v methanol. The membrane was blocked overnight at 4° C. in a cold-room in 5% non-fat dried milk in TBS-Tween pH 7.4 (50 mM Tris-HCl pH 7.4, 138 mM NaCl, 2.7 mM KCl (Fluka), 0.05% v/v Tween-20 (Sigma)). The membrane was then incubated at room temperature on a gyro-rocker SSL3 platform rocker (Stuart) for 1 h with a 1:1000 dilution of anti-poly histidine mouse IgG H1029 (Sigma) in TBS-Tween. After washing with TBS-Tween, the membrane was incubated for 2 h with a 1:2000 dilution of goat anti-mouse IgG-HRP conjugate A4416 (Sigma) in TBS-Tween. After washing with TBS-Tween, the blot was visualized using the Super Signal West Pico Chemiluminescent substrate (Thermo-Scientific) following the manufacturer's recommendations and BioMax XAR X-ray film (Kodak).

### Enzymatic Activity Assays

Prolyl and lysyl hydroxylase assays were performed substantially as described in Kivirikko et al. (1982) Methods Enzymol 82 Pt A, 245-304. All solutions were kept on ice. Fresh stocks of 2 mM FeSO₄ (Fluka), 20 mM ascorbate (Sigma), and 6 mM 2-oxoglutarate (Fluka) were prepared. Enzyme and acceptor substrate (either His-tag purified, E. coli expressed L71 Mimivirus collagen in MCAC10 buffer, or collagen-like peptide acceptors (GenScript) dissolved in ddH₂O (double-distilled water) were added to each reaction tube (either a 1.5 or 2 mL microcentrifuge tube). A master-mix of the remaining assay components was prepared, and aliquots of this were used to initiate each assay. The assay contained 50 nCi of [¹⁴C]-2-oxoglutarate (Perkin Elmer), 300 μM 2-oxoglutarate, 100 μM FeSO₄, 1 mM ascorbate, 50 mM Tris-HCl pH 7.4, and 100 μM DTT. When peptide acceptor substrates were used, the assay contained 600 μg/mL of peptide. Total assay volume was 100 μL with the master-mix component comprising no less than half the total volume. A small rectangular filter paper was soaked in NCS II Tissue Solubilizer (Amersham) and suspended from a small hook in a rubber stopper. The top was cut from the microcentrifuge tube containing the enzyme and acceptor substrate which was then carefully lowered into a 30 mL scintillation vial (VWR). The assay was initiated by addition of the master mix, and the vial was immediately closed with the stopper allowing the soaked filter paper to absorb any radioactive [¹⁴C]-CO₂ produced. The vial was incubated at 37° C. for 1 h. The assay was stopped with 100 μL of ice cold 1 M KH₂PO₄ administered into the reaction tube by a syringe and needle inserted through the stopper. The stopped assay was incubated for 30 min at room temperature, at which point the rubber stopper was removed and the filter paper transferred to a fresh scintillation vial. The filter paper was vortexed for approximately 5 s with 10 mL of IRGA-Safe Plus scintillation fluid (Perkin Elmer) and then measured in a Tri-Carb 2900TR scintillation counter (Packard). Mock assays contained no acceptor substrate.

### Amino Acid Analysis

Protein samples were hydrolysed by addition of 1 mL of 6 M HCL and incubated for 2 to 3 days at 105° C. The hydrolysate was dried in a Speedvac centrifuge, washed twice with 400 μl water and dissolved in 500 μl of borate buffer pH 11.4. 200 μL of hydrolysate was mixed with 200 μL of 6 mM FMOC in acetone and vortexed. After incubating for at least 40 min at room temperature, the derivatized amino acids were extracted 5 times with 600 μL of pentane. 400 μL of 25% v/v acetonitrile containing 25 mM boric acid were added to the pentane extracted FMOC-amino acids before injection into the HPLC system. Amino acid analysis utilizing HPLC was performed substantially as described in Schegg et al. (2009) Mol Cell Biol 29, 943-952. Up to 100 μL of sample was injected over an ODS Hypersil 150 mm×3 mm column with a 3 μm particle size (Thermo Scientific).

### Mass Spectrometry

His₁₀-tagged human collagen III co-expressed in E. coli BL21(DE3) with Mimivirus L593 prolyl-hydroxylase was purified by affinity chromatography with Ni-sepharose and then electrophoresed through a NuPAGE 4-12% Bis-Tris SDS-PAGE gel (Invitrogen). Subsequent to staining with Instant Blue Coomassie blue stain (Expedeon), the collagen-III band was excised and destained with ddH₂O following the manufacturers protocol. The band was destained in 100 mM ammonium bicarbonate/acetonitrile (1:1, vol/vol) then digested with freshly prepared trypsin (15 ng/μL) for 12 h at 37° C. The tryptic digest was analyzed using an Orbitrap mass spectrometer (Thermo Scientific). The spectra were selected from the Mascot generic file and imported into Excel. Assignments were made and protein peptide identifications statistically validated with the Protein Pilot version 3 software (Sciex) using the Paragon algorithm.

1.-20. (canceled)

21. A method of hydroxylating collagen comprising reacting collagen with an isolated prolyl or lysyl hydroxylase from Mimivirus comprising the sequence SEQ ID NO:1, SEQ ID NO:2 or SEQ ID NO:3, or a variant of such a protein in which one, two, three, four or five amino acids are exchanged by other naturally occurring amino acids.

22. The method according to claim 21, wherein the prolyl or lysyl hydroxylase from Mimivirus comprises the sequence SEQ ID NO:1.

23. The method according to claim 21, wherein the prolyl or lysyl hydroxylase from Mimivirus comprises the sequence SEQ ID NO:2.

24. The method according to claim 21, wherein the prolyl or lysyl hydroxylase from Mimivirus comprises the sequence SEQ ID NO:3.

25. The method according to claim 21 performed in situ in an E. coli host cell.

26. An isolated DNA comprising a DNA of the sequence SEQ ID NO:4, SEQ ID NO:5 or SEQ ID NO:6, or a variant of such DNA comprising variants of SEQ ID NO:4, SEQ ID NO:5 or SEQ ID NO:6 in which one or more nucleotides are replaced by other nucleotides in a triplet codon coding for the same amino acid as the original triplet codon, and/or one, two, three, four or five triplet codons are replaced by triplet codons coding for a different amino acid.

27. The isolated DNA according to claim 26 comprising a DNA of the sequence SEQ ID NO:4, SEQ ID NO:5 or SEQ ID NO:6.

28. A vector comprising a DNA according to claim 26.

29. The vector according to claim 28, which is a bicistronic vector comprising a DNA of the sequence SEQ ID NO:4 and of the sequence SEQ ID NO:6.

30. A host cell comprising a vector according to claim 28.

31. A host cell comprising a vector according to claim 29.

32. A host cell according to claim 30 expressing collagen, a protein comprising the sequence SEQ ID NO:1 and a protein comprising the sequence SEQ ID NO:3.

33. A host cell according to claim 31 expressing collagen, a protein comprising the sequence SEQ ID NO:1 and a protein comprising the sequence SEQ ID NO:3.

34. A method of manufacture of collagen in a host cell, comprising culturing a host cell expressing collagen according to claim 30 and isolating the collagen.

35. A method of manufacture of collagen in a host cell, comprising culturing a host cell expressing collagen according to claim 31 and isolating the collagen.

36. A method of gene therapy correcting lack of hydroxylated collagen, comprising administering a nucleotide according to claim 36 to a patient in need of hydroxylated collagen.

37. A method of wound healing and/or surgery, comprising administering to a patient in need thereof the collagen manufactured according to claim 32 in an amount effect for wound healing and/or surgery.

38. A method of growing tissue or organs in vitro and in vivo, comprising adding to the tissue or organs in vitro and/or in vivo the collagen manufactured according to claim 32 as scaffold to the growth medium.

39. A method of culturing cells with improved cell viability, comprising adding the collagen manufactured according to claim 32 as an additive to the cell culture medium.


SEQUENCE LISTING (22 sequences)

SEQ ID NO: 1 - protein, 222 aa, Acanthamoeba polyphaga mimivirus
MVLSKSCVSHFRNVGSLNSRDVNLKDDFSYANIDDPYNKPFVLNNLINPTKCQEIMQFANGKLFDSQVLSGTDKNIRNSQQMWISKNNPMVKPIFENICRQFNVPFDNAEDLQVVRYLPNQYYNEHHDSCCDSSKQCSEFIERGGQRILTVLIYLNNEFSDGHTYFPNLNQKFKPKTGDALVFYPLANNSNKCHPYSLHAGMPVTSGEKWIANLWFRERKFS

SEQ ID NO: 2 - protein, 242 aa, Acanthamoeba polyphaga mimivirus
MKTVTIITIIVVIIVVILIIMVLSKSCVSHFRNVGSLNSRDVNLKDDFSYANIDDPYNKPFVLNNLINPTKCQEIMQFANGKLFDSQVLSGTDKNIRNSQQMWISKNNPMVKPIFENICRQFNVPFDNAEDLQVVRYLPNQYYNEHHDSCCDSSKQCSEFIERGGQRILTVLIYLNNEFSDGHTYFPNLNQKFKPKTGDALVFYPLANNSNKCHPYSLHAGMPVTSGEKWIANLWFRERKFS

SEQ ID NO: 3 - protein, 895 aa, Acanthamoeba polyphaga mimivirus
MISRTYVINLARRPDKKDRILAEFLKLKEKGVELNCVIFEAVDGNNPEHLSRFNFKIPNWTDLNSGKPMTNGEVGCALSHWSVWKDVVDCVENGTLDKDCRILVLEDDVVFLDNFMERYQTYTSEITYNCDLLYLHRKPLNPYTETKISTHIVKPNKSYWACAYVITYQCAKKFMNANYLENLIPSDEFIPIMHGCNVYGFEKLFSNCEKIDCYAVQPSLVKLTSNAFNDSETFHSGSYVPSNKFNFDTDKQFRIVYIGPTKGNSFHRFTEYCKLYLLPYKVIDEKETNDFVSLRSELQSLSEQDLNTTLMLVVSVNHNDFCNTIPCAPTNEFIDKYKQLTTDTNSIVSAVQNGTNKTMFIGWANKISEFINHYHQKLTESNAETDINLANLLLISSISSDFNCVVEDVEGNLFQLINEESDIVFSTTTSRVNNKLGKTPSVLYANSDSSVIVLNKVENYTGYGWNEYYGYHVYPVKFDVLPKIYLSIRIVKNANVTKIAETLDYPKELITVSISRSEHDSFYQADIQKFLLSGADYYFYISGDCIITRPTILKELLELNKDFVGPLMRKGTESWTNYWGDIDPSNGYYKRSFDYFDIIGRDRVGCWNVPYLASVYLIKKSVIEQVPNLFTENSHMWNGSNIDMRLCHNLRKNNVFMYLSNLRPYGHIDDSINLEVLSGVPTEVTLYDLPTRKEEWEKKYLHPEFLSHLQNFKDFDYTEICNDVYSFPLFTPAFCKEVIEVMDKANLWSKGGDSYFDPRIGGVESYPTQDTQLYEVGLDKQWHYVVFNYVAPFVRHLYNNYKTKDINLAFVVKYDMERQSELAPHHDSSTYTLNIALNEYGKEYTAGGCEFIRHKFIWQGQKVGYATIHAGKLLAYHRALPITSGKRYILVSFVN

SEQ ID NO: 4 - DNA, 669 bp, Acanthamoeba polyphaga mimivirus
atggtattgtcaaaatcttgtgtgtcccattttagaaatgtcggtagtttgaattcacgggatgtgaatttaaaagatgatttttcgtacgccaatattgatgatccttataataaaccatttgttttgaataatttgattaatccgaccaaatgtcaagaaattatgcaatttgccaatggaaaactttttgattcacaagttttaagtggtactgataaaaatattcgcaatagtcaacaaatgtggatatctaaaaataatcctatggtcaaacctatttttgaaaatatatgtagacaatttaatgttccatttgataatgctgaagatttgcaagttgttcgttatttacctaatcaatattataatgaacaccatgattcatgctgtgatagtagtaaacaatgtagtgaatttattgaaagaggtggtcaaagaattctaaccgtactaatttatctcaataatgaattttctgatggtcatacttatttccctaatttgaatcaaaaattcaaaccaaaaacaggagatgcactagtattttaccctttagctaataatagtaataaatgtcatccatattctttacatgctggaatgcctgttactagtggagaaaaatggattgccaatttatggtttagagaacgaaaattttcctaa

SEQ ID NO: 5 - DNA, 729 bp, Acanthamoeba polyphaga mimivirus
atgaaaactgtgactatcattacaataattgttgtaattattgttgttattttgattataatggtattgtcaaaatcttgtgtgtcccattttagaaatgtcggtagtttgaattcacgggatgtgaatttaaaagatgatttttcgtacgccaatattgatgatccttataataaaccatttgttttgaataatttgattaatccgaccaaatgtcaagaaattatgcaatttgccaatggaaaactttttgattcacaagttttaagtggtactgataaaaatattcgcaatagtcaacaaatgtggatatctaaaaataatcctatggtcaaacctatttttgaaaatatatgtagacaatttaatgttccatttgataatgctgaagatttgcaagttgttcgttatttacctaatcaatattataatgaacaccatgattcatgctgtgatagtagtaaacaatgtagtgaatttattgaaagaggtggtcaaagaattctaaccgtactaatttatctcaataatgaattttctgatggtcatacttatttccctaatttgaatcaaaaattcaaaccaaaaacaggagatgcactagtattttaccctttagctaataatagtaataaatgtcatccatattctttacatgctggaatgcctgttactagtggagaaaaatggattgccaatttatggtttagagaacgaaaattttcctaa

SEQ ID NO: 6 - DNA, 2688 bp, Acanthamoeba polyphaga mimivirus
atgattagtagaacttatgtaattaatcttgctagacgacctgataagaaagatcgtattcttgcggaattcctcaaactcaaagaaaaaggtgttgagcttaattgtgtaatttttgaagctgttgatggaaataatccggaacatttatcgagatttaatttcaagattcctaattggactgacttgaattcaggtaagccaatgactaatggagaagttggttgtgcattgagtcattggtctgtgtggaaagatgttgtggattgtgtagaaaatggtactctagataaagattgtcgcattcttgtattggaagatgatgttgtttttcttgataattttatggaacgatatcaaacttatacttctgaaattacttacaattgtgatctactctacctgcatagaaaacctttgaatccctatactgaaacaaaaatctctactcatattgtcaaaccaaataaaagttactgggcttgcgcatatgtcattacttatcaatgtgccaaaaaattcatgaatgctaattatttagaaaacctaattccgagtgatgaatttattccgattatgcatgggtgtaatgtctatggttttgaaaaattattttccaattgtgaaaaaatagattgttacgcagttcaacctagtctcgtaaaattaacatctaatgcttttaatgatagcgaaacatttcattcgggttcttatgtaccaagtaataaatttaattttgatactgataaacagtttagaattgtatatattggacccactaaaggtaattcattccatagatttactgaatattgtaaactttatttattaccttataaagtgatcgatgaaaaagaaaccaatgattttgtttcacttagatcagaacttcaatctcttagtgaacaagatctcaataccacactcatgttggttgtttcagtaaatcacaatgatttttgtaatactattccatgtgcaccaaccaatgaattcattgacaagtataaacaattaacaactgatactaattctattgttagtgctgttcaaaatggaactaataagactatgttcatcggttgggccaataaaattagtgaatttattaatcattatcatcaaaaacttactgaatctaatgccgaaacagatattaatctagctaatttattacttataagttctatttcatccgattttaattgtgttgtagaagatgttgaaggtaatttgttccaattaattaacgaagaatcagatattgtatttagtacaacaacttccagagtcaacaacaaattaggtaaaacaccaagcgttttgtatgccaattctgattcttctgtgattgtacttaataaagtagaaaattatacaggttatggttggaatgaatattatggttatcatgtttatccagttaaatttgatgttcttccaaaaatctatctttcaattcgcattgtaaagaatgcaaatgttactaaaattgctgaaactcttgactatccaaaagaattaatcactgtttcgatcagtcgatcagaacatgatagtttttatcaagctgatattcagaaattcttattgagtggtgctgattattatttttacatttcaggagattgtatcattactcgaccaactattctaaaagaacttctggaactcaataaagattttgtaggtcctctcatgcgtaagggtactgaatcatggactaactattggggtgatatcgatccttctaatggttattacaaaagatcatttgattattttgatattattggtagagatagagttggttgttggaatgtaccatatctggcaagcgtctatttaattaaaaaatctgtcattgaacaagttccaaatttgtttactgaaaatagtcacatgtggaatggtagtaatattgatatgagattatgtcacaatcttcgtaaaaataatgtattcatgtatttgagtaatctccgtccttatggacacattgatgattctattaacctggaagttctttctggtgttcctaccgaagttactctttatgatcttccaacgcgaaaagaagaatgggagaaaaagtatcttcatcccgaatttttgagtcatttacaaaattttaaagattttgattatactgaaatttgtaacgatgtttatagtttcccactttttacacctgctttctgtaaagaggttattgaagttatggataaagccaatttgtggtctaaaggtggtgattcttattttgatccaagaattggtggtgttgaatcttatcctactcaagatactcaactgtatgaggtaggattagataaacaatggcattatgtcgttttcaattatgttgcaccatttgtacgtcatttatacaataattataaaaccaaagatattaatttagcttttgttgttaaatatgatatggaaagacaatctgaattggctcctcatcatgattcttccacatatactttaaatattgcacttaatgaatacggtaaagaatatacggccggaggttgcgagttcattcgtcataaatttatctggcaaggacaaaaagttggttacgctacaattcacgctggaaaactattggcatatcatcgagctcttccaattacttccggtaaaagatatattttagtgtcttttgttaattaa

SEQ ID NO: 7 - protein, 21 aa, Artificial
  Prolyl hydroxylase substrate
GPPGPPGPPGPPGPPGPPGPP

SEQ ID NO: 8 - protein, 12 aa, Artificial
  Lysyl hydroxylase substrate
GDKGDKGDKGDK

SEQ ID NO: 9 - protein, 12 aa, Artificial
  Lysyl hydroxylase substrate
GIKGIKGIKGIK

SEQ ID NO: 10 - protein, 12 aa, Artificial
  Lysyl hydroxylase substrate
GIMGYKGEKGEI

SEQ ID NO: 11 - protein, 12 aa, Artificial
  Lysyl hydroxylase substrate
GDKGDVGDKGDV

SEQ ID NO: 12 - protein, 12 aa, Artificial
  Lysyl hydroxylase substrate
GDIGSKGETGNK

SEQ ID NO: 13 - protein, 12 aa, Artificial
  Lysyl hydroxylase substrate
GTKGETGLKGII

SEQ ID NO: 14 - DNA, 32 bp, Artificial
  Primer
tgacctcgaggtattgtcaaaatcttgtgtgt

SEQ ID NO: 15 - DNA, 33 bp, Artificial
  Primer
cagggatccattttgtgttaaaaaaattttagg

SEQ ID NO: 16 - DNA, 34 bp, Artificial
  Primer
tgacctcgagaaaactgtgactatcattacaata

SEQ ID NO: 17 - DNA, 30 bp, Artificial
  Primer
ggcaagctttggtgagctttgtgcaaaagg

SEQ ID NO: 18 - DNA, 38 bp, Artificial
  Primer
atcgcggccgcttataaaaagcaaacagggccaacgtc

SEQ ID NO: 19 - DNA, 34 bp, Artificial
  Primer
gacccatgggatccattagtagaacttatgtaat

SEQ ID NO: 20 - DNA, 35 bp, Artificial
  Primer
gtcactagtttaattaacaaaagacactaaaatat

SEQ ID NO: 21 - DNA, 31 bp, Artificial
  Primer
tgacctcgagattagtagaacttatgtaatt

SEQ ID NO: 22 - DNA, 29 bp, Artificial
  Primer
cagggatccgtccaataaagtgtatcaac